(19) United States
(12) Patent Application Publication
(10) Pub. No.: US 2014/0079813 A1
(43) Pub. Date: Mar. 20, 2014

(54) Genes Associated With Post Relapse Survival And Uses Thereof

(71) Applicants: Joshua Epstein, Little Rock, AR (US); Shmuel Yaccoby, Little Rock, AR (US); John D. Shaughnessy, JR., Roland, AR (US); Barthel Barlogie, Little Rock, AR (US); Igor Entin, Little Rock, AR (US)

(72) Inventors: Joshua Epstein, Little Rock, AR (US); Shmuel Yaccoby, Little Rock, AR (US); John D. Shaughnessy, JR., Roland, AR (US); Barthel Barlogie, Little Rock, AR (US); Igor Entin, Little Rock, AR (US)

(21) Appl. No.: 14/114,406

(22) PCT Filed: Apr. 27, 2012
(86) PCT No.: PCT/US2012/035494

§ 371 Date: Oct. 28, 2013

Related U.S. Application Data
(63) Continuation of application No. 13/068,008, filed on Apr. 29, 2011.

(51) Int. Cl. C12Q 1/68; A61K 31/704; A61K 31/573; A61K 33/24; A61K 31/454; A61K 31/4965

(52) U.S. Cl. CPC C12Q 1/6886; A61K 31/454; A61K 31/4965; A61K 31/573; A61K 33/24; A61K 31/704
USPC 424/649; 435/6.12; 506/9; 514/323; 514/255.06; 514/171

(57) ABSTRACT

Provided are methods, systems and kits for predicting post-relapse survival of a cancer patient and for identifying cancer genes predictive of the post-relapse survival of the patient. Values representing gene expression levels of a group of genes associated with survival of the cancer cells are determined using gene expression profiling platforms and a plurality of probe sets that hybridize to one or more of the genes in the group. A predictive model establishes a predictive value based on the weighted contribution of each gene associated with survival of the cancer cells to risk of death for the cancer patient and imports expression values of the genes in the group that is indicative of a risk of death for the relapsed patient. Using global gene expression profiling and statistical analysis, expression of cancer cell genes at baseline and at first relapse that are involved in interaction of cancer cells with cells in their microenvironment, can be used to identify genes that are predictive of post-relapse survival.

### RELATED APPLICATION(S)

This application is a continuation-in-part and claims priority to Ser. No. 13/068,008, filed 29 Apr. 2011.

The entire teachings of the above application(s) are incorporated herein by reference.

### GOVERNMENT SUPPORT

This invention was made with government support under grants CA-113992, CA-093897 and CA-055819 awarded by the National Cancer Institute. The government has certain rights in the invention.

### BACKGROUND OF THE INVENTION

1. Field of the Invention

The present invention relates generally to the fields of gene expression profiling and cancer prognosis. More specifically, the present invention discloses methods and systems for a predictive model utilizing a group of genes associated with survival of cancer cells to predict post-relapse survival of a cancer patient.

2. Background Description

Multiple myeloma is unique among the hematological malignancies in that in the vast majority of patients its growth is restricted to the bone marrow. Development of myeloma is intimately associated with osteolytic bone disease in over 80% of patients, as a result of inhibition of osteoblast differentiation and stimulation of osteoclastogenesis. Myeloma is also unique among all tumors that metastasize to the bone marrow and cause osteolysis; myeloma-induced osteolytic lesions do not repair, even after many years of complete remission. Myeloma associated lytic bone disease results from disruption of the RANKL/OPG axis, an effect likely mediated by myeloma-cell secretion of the Wnt signaling inhibitor Dickkopf-1 (DKK-1). By inhibiting Wnt signaling, DKK-1 blocks the differentiation of bone marrow mesenchymal cells (MSC) to osteoblasts, increasing expression of RANKL and reducing expression of OPG, resulting in stimulation of osteoclast formation and activity. Indeed, in myeloma patients the soluble RANKL/OPG ratios correlate with the extent of osteolytic bone disease.

Progression of the pre-malignant plasma cell dyscrasia monoclonal gammopathy of unknown significance (MGUS) to myeloma is preceded by changes in bone turnover rates; an initial coupled increase in both osteoblast and osteoclast activity is followed with disease progression by decreased osteoblast activity while osteoclast activity remains elevated, leading to osteolytic bone disease. Myeloma cell dependence on the bone marrow microenvironment and on the changes they induce in the bone marrow is also evident in a SCID-hu model for primary human myeloma, where growth of freshly obtained primary myeloma cells is restricted to the human bone implants. Using this model, it was demonstrated that myeloma growth is dependent on osteoclast activity. This observation was reproduced in culture, where osteoclasts supported myeloma cells survival.

In contrast to osteoclasts, which always support myeloma cell survival in vitro and in vivo, osteoblast effects in the SCID-hu model varied from none to increase in bone formation associated with inhibition of myeloma growth. In co-cultures, osteoblasts differentiated from mesenchymal cells inhibited survival of freshly isolated myeloma cells, suggesting that inhibition of osteoblast differentiation supports myeloma cell survival. The interactions between myeloma cells and bone cells, as well as the molecular consequences of myeloma cell interactions with osteoclasts and mesenchymal cells are not well understood.

While new myeloma therapies have achieved high rates of complete remission and near-complete remission (1), relapses are common, and most patients experience short post-relapse survival, Thus, there is a recognized need in the art to better able to predict the likelihood of survival of a myeloma patients after relapse of the cancer. The prior art is deficient in the identification of genes that are associated with the survival of myeloma cells and are potential targets for interventions, and methods and systems for predicting post relapse survival of myeloma patients. The present invention fulfills this longstanding need and desire in the art.

### SUMMARY OF THE INVENTION

The present invention is directed to a method for predicting post-relapse survival of a cancer patient in a state of relapse. The method comprises importing individual values for gene expression of a group of genes associated with survival of cancer cells obtained from the cancer patient after relapse of the cancer into a predictive model, which is a statistical model. Using the predictive model, a predictive value, based on the weighted contribution of each gene to a risk of death for the cancer patient and the imported expression values of the genes in the group, is established that is indicative of a risk of death for the relapsed cancer patient, thereby predicting post-relapse survival of the cancer patient.

The present invention is directed to a related method for predicting post-relapse survival of a multiple myeloma patient in a state of relapse. The method comprises hybridizing nucleic acids obtained from multiple myeloma cells in the relapsed patient to one or more platforms comprising probe sets hybridizable to one or more genes in a group of genes associated with survival of the multiple myeloma cells and converting intensity of a signal generated upon hybridization to the value of gene expression for each gene in the group. Values for gene expression of each gene in the group are imported into a predictive model, which is a statistical model. Using the predictive model a predictive value, based on the weighted contribution of each gene to risk of death for the relapsed multiple myeloma patient and the imported expression values of the genes in the group, is established that is indicative of a risk of death for the relapsed patient, thereby predicting post-relapse survival of the cancer patient.

The present invention also is directed to another method for predicting post-relapse survival of a cancer patient in a state of relapse. The method comprises measuring the level of gene expression of a group of multiple myeloma genes comprising at least CCNE2, PECAM1, HMOX1, and CISH; optionally further comprising HBEGF, JUN, SIX5, and DUSP1; optionally further comprising BMP6, FOSB, and LIME1; optionally further comprising multiple myeloma genes BIRC3, FER1L4, KLHL21, MAFF, SOCS3, and TSC22D3 from multiple myeloma cells obtained from the patient before the start of a treatment regimen for the cancer, and measuring the level of gene expression of the genes obtained from the patient after relapse of the multiple myeloma. The expression level of each gene before treatment is compared with the expression level of each corresponding gene after relapse where a decrease in expression of PECAM1, HMOX1, CISH, SIX5, BMP6, JUN, FOSB and DUSP1, and an increase in expression of LIME1, CCNE2, HBEGF has a statistically significant correlation with post-relapse survival of the myeloma cells in the patient and is predictive of a low likelihood of survival of the patient. The present invention is directed to a related method where the group of myeloma genes further comprises. BIRC3, FER1L4, TSC22D3, MAFF, SOCS3, and KLHL21. A decrease in expression level of BIRC3, FER1L4, and TSC22D3, and an increase in expression level of MAFF, SOCS3 and KLHL21 are predictors of a likelihood of a shorter survival of the patient.

The present invention is directed further to a system for predicting post-relapse survival of a multiple myeloma patient in a state of relapse. The system comprises one or more platforms having probe sets hybridizable to one or more multiple myeloma genes in a group comprising at least CCNE2, PECAM1, HMOX1, and CISH; optionally further comprising HBEGF, JUN, SIX5, and DUSP1; optionally further comprising BMP6, FOSB, and LIME1; optionally further comprising multiple myeloma genes BIRC3, FER1L4, KLHL21, MAFF, SOCS3, and TSC22D3 and a signal processor configured to convert intensity of a hybridization signal to a value of gene expression for each gene in the group. A predictive model configured to import the gene expression values comprises a calculator that uses a summation function of an assigned risk of death for each gene in the group to calculate the risk of death of the relapsed patient, where risk for each gene is assigned on a sliding scale and is a product of each gene's weight in determining risk of death and the imported expression value of the gene.

The present invention is directed further still to a kit for predicting post-relapse survival of a multiple myeloma patient in a state of relapse. The kit comprises the predictive model of the system and is tangibly stored on a computer storage medium. The present invention is directed to a related kit further comprising a platform that has a plurality of probes hybridizable to one or more of multiple myeloma genes CCNE2, PECAM1, HMOX1, and CISH; optionally further comprising HBEGF, JUN, SIX5, and DUSP1; optionally further comprising BMP6, FOSB, and LIME1. The present invention is directed to another related kit that further comprises a plurality of probes hybridizable to one or more of multiple myeloma genes BIRC3, FER1L4, TSC22D3, MAFF, SOCS3 and KLHL21.

The present invention is directed further still to a method for identifying cancer genes predictive of post-relapse survival for a cancer patient. The method comprises co-culturing cancer cells with cells that interact with the cancer cells in their microenvironment and performing a first global gene expression profiling on the cancer cells before co-culture, and a second global gene expression profiling after co-culture. From comparing the first and the second gene expression profile, a set of genes differentially expressed after co-culture are identified via statistical analysis. A third global gene expression profiling is performed on post-relapse cancer cells obtained from relapsed cancer patients and post-relapse genes whose expression was differentially changed are identified via statistical analysis of the third expression profile. A comparison between post-relapse expression of the genes whose expression differentially changed after co-culture and duration of survival of the post-relapse cancer patients identifies the cancer genes predictive of post-relapse survival of the cancer patient. The present invention is directed to a related method further comprising performing a multivariate permutation test to eliminate genes with a higher than a pre-determined false positive change in expression. The present invention is directed to another related method further comprising identifying networks of interrelated genes among the differentially expressed gene set to further narrow the genes comprising the same.

Other and further aspects, features, and advantages of the present invention will be apparent from the following description of the presently preferred embodiments of the invention. These embodiments are given for the purpose of disclosure.

### BRIEF DESCRIPTION OF DRAWINGS

So that the matter in which the above-recited features, advantages and objects of the invention, as well as others which will become clear, are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIG. 1 shows purity of myeloma cells recovered from co-culture with osteoclasts. Myeloma plasma cells were recovered from co-culture. The recovered cells were reacted with monoclonal antibodies to CD38 (PE conjugated) and CD45 (FITC conjugated) and analyzed by flow cytometry. The purity was routinely >95%.

FIGS. 2A-2E are five networks depicting interrelationships among 54 multiple myeloma plasma cell genes with high probability IPA scores of the 58 genes whose gene expression changes following co-culture with mesenchymal stem cells was similar to that following co-culture with osteoclasts.

FIGS. 3A-3C show a Kaplan-Meyer analysis of post relapse survival of patients. 127 patients that were treated with total therapy 2 were used as a training set, from which the predictive model was developed, where expression 11 genes, represented by 13 probe sets in Table 6 (FIG. 3A) predicted survival of the patients. The 11 genes in Table 6 predicted survival of 32 patients who relapsed on total therapy 3 protocol (FIG. 3B) and 98 patients who relapsed after various treatment protocols (FIG. 3C). Risk was assigned by BRB ArrayTools software using expression signal of 72 probesets identified by co-culture experiments. Expression signals were dichotomized at the median.

FIGS. 4A-4D are bar graphs summarizing the correlation between microarray-based gene expression profiling (GEP; FIGS. 4A and 4C) and quantitative real-time polymerase chain reaction (qRT-PCR; FIGS. 4B and 4D) for PECAM (FIGS. 4A and 4B) and CCNE2 (FIGS. 4C and 4D) in 10 patients. The mRNA used in both the GEP and qRT-PCR was from a single preparation from isolated plasma cells.

FIGS. 5A-5C show a Kaplan-Meyer analysis of post relapse survival of patients evaluated by the 33 probeset model in Table 7. 127 patients were subject to Total Therapy II (TT2, the training set; FIG. 5A), 98 patients were treated by various protocols (FIG. 5B), and 32 patients were treated by Total Therapy III (TT3; FIG. 5C).

### DETAILED DESCRIPTION OF THE INVENTION

As used herein, the following terms and phrases shall have the meanings set forth below. Unless defined otherwise, all technical and scientific terms used herein have the same meaning as commonly understood to one of ordinary skill in the art.

As used herein, the term, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof. The terms “comprise” and “comprising” are used in the inclusive, open sense, meaning that additional elements may be included.

As used herein, the term “or” in the claims refers to “and/or” unless explicitly indicated to refer to alternatives only or the alternatives are mutually exclusive, although the disclosure supports a definition that refers to only alternatives and “and/or”.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

As used herein, the term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent” which is a biologically, physiologically, or pharmacologically active substance (or substances) that acts locally or systemically in a subject.

A “patient,” “individual,” “subject” or “host” refers to either a human or a non-human animal, e.g., non-human mammals. The term “mammal” is known in the art, and exemplary mammals include humans, primates, bovines, porcines, canines, felines, and rodents, e.g., mice and rats.

The invention provides, inter alia, methods, systems, and kits for predicting post-relapse survival of a cancer patient, particularly a multiple myeloma patient, using groups of multiple myeloma genes. In one embodiment, the group of multiple myeloma genes consists of, consists essentially of, or comprises CCNE2, PECAM1, HMOX1, and CISH. In certain embodiments, the group of multiple myeloma genes further comprises HBEGF, JUN, SIX5, and DUSP1 (i.e. the group consists of, consists essentially of, or comprises CCNE2, PECAM1, HMOX1, CISH, HBEGF, JUN, SIX5, and DUSP1). In more particular embodiments, the group of multiple myeloma genes further comprises BMP6, FOSB, and LIME1 (i.e. the group consists of, consists essentially of, or comprises CCNE2, PECAM1, HMOX1, CISH, HBEGF, JUN, SIX5, DUSP1, BMP6, FOSB, and LIME1). In still more particular embodiments, the group of multiple myeloma genes further comprises BIRC3, FER1L4, TSC22D3, MAFF, SOCS3, and KLHL21 (i.e., the group consists of, consists essentially of, or comprises CCNE2, PECAM1, HMOX1, CISH, HBEGF, JUN, SIX5, DUSP1, BMP6, FOSB, and LIME1, BIRC3, FER1L4, TSC22D3, MAFF, SOCS3, and KLHL21). In other embodiments, the group of multiple myeloma genes consists of, consists essentially of, or comprises the genes in Table 7 (i.e. OSBPL10, CCNE2, UBE2D1, PECAM1, APOE, ATP6AP2, PTPRG, ZNF267, PECAM1, HMOX1, SOD2, TXNRD1, CD58, MS4A7, PPBP, EPAS1, FLNA, EPHB1, PLA2G7, CISH, SQSTM1, KLHL14, ATP6V1C1, FAM13A, PECAM1, SCD5, GM2A, CREB3L2, CD163, CPM, IDUA, SPAG4, and Clorf38—optionally, e.g., using the particular probes recited in Table 7). In still other embodiments, the group of multiple myeloma genes consists of, consists essentially of, or comprises the genes in Table 2 (i.e. AMPD 1, ANPEP, BIRC3, BMP6, BTLA, CBFA2T3, CCNE2, CCR2, CCR7, CD44, CD48, CD82, CEP170, CHST11, CISH, CITED2, DUSP1, EPB41L3, FAM129B, FER1L4, FOS, FOSB, GLA, GPRC5D, HBEGF, HMOX1, ICAM1, IFI30, IL8, ISG20, JUN, KANK1, KLF2, KLHL21, LIME1, LRRN1, MAFF, MMP14, MT1E, MT1H, PECAM1, PLAC8, PLAU, PLAUR, PRRG4, SAT1, SGK1, SIX5, SLC25A19, SLC2A4RG, SOCS3, SPHK1, TGM2, TMEM49, TNFAIP2, TNFRSF17, TSC22D3, and UGT2B17—optionally, e.g., using the particular probes recited in Table 2).

Any of the following methods, system, or kits may be readily adapted to use the different groups of multiple myeloma genes above. The predictive values of the genes can be evaluated in a variety of ways, e.g., according to the hazard ratios in any of Tables 4 or 5 (e.g., for genes with a hazard ratio>1, increased expression correlates with low likelihood of survival and genes with a hazard ratio<1, increased expression correlates with high survival) or the weights in Table 7 (positive values indicate genes where an increase in expression is associated with low survival, negative values indicate genes where an increase in expression is associated with high survival). Expression levels are evaluated by any suitable means, such as PCR, microarray, or sequencing, including combinations of these. In particular embodiments, the levels are measured using microarray, such as oligonucleotide microarrays, including AFFYMETRIX® arrays, such as U133Plus2.0 microarrays. In still more particular embodiments, expression levels are evaluated on an oligonucleotide microarray using the probes provided in Tables 1-7. Levels are typically measured in multiple myeloma plasma cells. In particular embodiments, the multiple myeloma plasma cells are CD138+. The cells are isolated at any time, but in particular embodiments are isolated at about the time of first relapse, e.g., within about 1, 2, 3, 4, 5, 6, 7, 8, 9, or 10 days of the first clinical signs of relapse, or in some embodiments, within about 1, 2, 3, 4, or 5 weeks, or within about 1, 2, 3, 4, 5, or 6 months of the first clinical signs of relapse.

In one embodiment of the present invention there is provided a method for predicting post-relapse survival of a relapsed cancer patient (e.g., a cancer patient in a state of relapse), comprising importing individual values for gene expression of a group of genes associated with survival of cancer cells obtained from the cancer patient after relapse of the cancer into a predictive model, where the predictive model is a statistical model; and establishing, with the predictive model, a predictive value based on the weighted contribution of each gene to risk of death for the cancer patient and the imported expression values of the genes in the group that is indicative of a risk of death for the relapsed cancer patient, thereby predicting post-relapse survival of the cancer patient.

In this embodiment obtaining values for gene expression of the genes in the group may comprise hybridizing nucleic acids obtained from the cancer cells under standard conditions to one or more platforms comprising probe sets hybridizable to one or more genes in the group; and converting intensity of a signal generated upon hybridization to the value of gene expression for each gene in the group. Also, in this embodiment establishing the predictive value may comprise summing the products of the weighted risk of each gene in the group in the predictive model and the imported expression level for each gene in the group. Weighted risk comprises a coefficient for each gene in the group where the coefficient is representative of each gene's weight contribution to a risk of death based on a hazard ratio for a 2-fold increase in gene expression such that, if the hazard is higher than 1, increased expression correlates to a higher risk of death and, if the hazard ratio is lower than 1, increased expression indicates a lower risk of death.

In an aspect of this embodiment the genes in the group of multiple myeloma genes consist of, consist essentially of, or comprise CCNE2, PECAM1, HMOX1, and CISH; optionally further comprising HBEGF, JUN, SIX5, and DUSP1; optionally further comprising BMP6, FOSB, and LIME1. Further to this aspect the genes in the group further comprises multiple myeloma genes BIRC3, FER1L4, KLHL21, MAFF, SOCS3, and TSC22D3. In all aspects of this embodiment the cancer may be multiple myeloma. In another related aspect, the genes in the group of multiple myeloma genes consists of, consists essentially of, or comprises multiple myeloma genes OSBPL10, CCNE2, UBE2D1, PECAM1, APOE, ATP6AP2, PTPRG, ZNF267, PECAM1, HMOX1, SOD2, TXNRD1, CD58, MS4A7, PPBP, EPAS1, FLNA, EPHB1, PLA2G7, CISH, SQSTM1, KLHL14, ATP6V1C1, FAM13A, PECAM1, SCD5, GM2A, CREB3L2, CD163, CPM, IDUA, SPAG4, and Clorf38. In yet another related aspect, the group of multiple myeloma genes consists of, consists essentially of, or comprises multiple myeloma genes AMPD1, ANPEP, BIRC3, BMP6, BTLA, CBFA2T3, CCNE2, CCR2, CCR7, CD44, CD48, CD82, CEP170, CHST11, CISH, CITED2, DUSP1, EPB41L3, FAM129B, FER1L4, FOS, FOSB, GLA, GPRC5D, HBEGF, HMOX1, ICAM1, IFI30, IL8, ISG20, JUN, KANK1, KLF2, KLHL21, LIME1, LRRN1, MAFF, MMP14, MT1E, MT1H, PECAM1, PLAC8, PLAU, PLAUR, PRRG4, SAT1, SGK1, SIX5, SLC25A19, SLC2A4RG, SOCS3, SPHK1, TGM2, TMEM49, TNFAIP2, TNFRSF17, TSC22D3, and UGT2B1.

In a related embodiment there is provided a method for predicting post-relapse survival of a relapsed multiple myeloma patient, comprising hybridizing nucleic acids obtained from multiple myeloma cells in the relapsed patient to one or more platforms comprising probe sets hybridizable to one or more genes in a group of genes associated with survival of the multiple myeloma cells; converting intensity of a signal generated upon hybridization to the value of gene expression for each gene in the group; importing values for gene expression of each gene in the group into a predictive model, where the predictive model is a statistical model; and establishing, with the predictive model, a predictive value based on the weighted contribution of each gene to risk of death for the relapsed multiple myeloma patient and the imported expression values of the genes in the group that is indicative of a risk of death for the relapsed patient, thereby predicting post-relapse survival of the cancer patient. In this embodiment the steps for establishing the predictive value, the weighted risk and the myeloma genes are generally described supra.

In another embodiment of the present invention there is provided a method for predicting post-relapse survival of a relapsed multiple myeloma patient, comprising measuring the level of gene expression of a group of multiple myeloma genes consisting of, consisting essentially of, or comprising at least CCNE2, PECAM1, HMOX1, and CISH; optionally further comprising HBEGF, JUN, SIX5, and DUSP1; optionally further comprising BMP6, FOSB, and LIME1 from multiple myeloma cells obtained from the patient before start of a treatment regimen for the cancer; measuring the level of gene expression of the genes obtained from the patient after relapse of the multiple myeloma; and comparing the expression level of each gene before treatment with the expression level of each corresponding gene after relapse; wherein a decrease in expression of PECAM1, HMOX1, CISH, SIX5, BMP6, JUN, FOSB, and DUSP1 and an increase in expression of LIME1, CCNE2, and HBEGF has a statistically significant correlation with post-relapse survival of the myeloma cells in the patient and is predictive of a low survival rate of the patient.

Further to this embodiment the group of multiple myeloma genes may comprise additional genes BIRC3, FER1L4, TSC22D3, MAFF, SOCS3, and KLHL21 and where decrease in expression level of BIRC3, FER1L4, and TSC22D3, and an increase in expression level of MAFF, SOCS3 and KLHL21 are predictors of shorter survival of the patient. In both embodiments measuring a level of gene expression of the genes in the group comprises hybridizing nucleic acids obtained from the myeloma cells to one or more platforms comprising probe sets hybridizable to one or more genes in the group; and converting intensity of a signal generated upon hybridization to the value of gene expression for each myeloma gene in the group.

In yet another embodiment the present invention provides a system for predicting post-relapse survival of a multiple myeloma patient in a state of relapse, comprising one or more platforms having probe sets hybridizable to one or more multiple myeloma genes in a group consisting of, consisting essentially of, or comprising at least CCNE2, PECAM1, HMOX1, and CISH; optionally further comprising HBEGF, JUN, SIX5, and DUSP1; optionally further comprising BMP6, FOSB, and LIME1; a signal processor configured to convert intensity of a hybridization signal to a value of gene expression for each gene in the group; and a predictive model configured to import the gene expression values and comprising a calculator that uses a summation function of an assigned risk of death for each gene in the group to calculate the risk of death of the relapsed patient, wherein risk for each gene is assigned on a sliding scale and is a product of each gene's weight in determining risk of death and the imported expression value of the gene.

Further to this embodiment the group of multiple myeloma genes may comprise additional genes BIRC3, FER1L4, TSC22D3, MAFF, SOCS3, and KLHL21. In both embodiments the predictive model comprises a computer program product tangibly stored in a computer memory or computer storage medium and configured to be executed by a processor. The predictive model comprises a coefficient, as described supra. In another related aspect, the probe sets are hybridizable to a group of multiple myeloma genes consists of, consists essentially of, or comprises multiple myeloma genes OSBPL10, CCNE2, UBE2D1, PECAM1, APOE, ATP6AP2, PTPRG, ZNF267, PECAM1, HMOX1, SOD2, TXNRD1, CD58, MS4A7, PPBP, EPAS1, FLNA, EPHB1, PLA2G7, CISH, SQSTM1, KLHL14, ATP6V1C1, FAM13A, PECAM1, SCD5, GM2A, CREB3L2, CD163, CPM, IDUA, SPAG4, and Clorf38. In yet another related aspect, the probe sets are hybridizable to a group of multiple myeloma genes consists of, consists essentially of, or comprises multiple myeloma genes AMPD1, ANPEP, BIRC3, BMP6, BTLA, CBFA2T3, CCNE2, CCR2, CCR7, CD44, CD48, CD82, CEP170, CHST11, CISH, CITED2, DUSP1, EPB41L3, FAM129B, FER1L4, FOS, FOSB, GLA, GPRC5D, HBEGF, HMOX1, ICAM1, IFI30, IL8, ISG20, JUN, KANK1, KLF2, KLHL21, LIME1, LRRN1, MAFF, MMP14, MT1E, MT1H, PECAM1, PLAC8, PLAU, PLAUR, PRRG4, SAT1, SGK1, SIX5, SLC25A19, SLC2A4RG, SOCS3, SPHK1, TGM2, TMEM49, TNFAIP2, TNFRSF17, TSC22D3, and UGT2B1.

In yet another embodiment the present invention provides a kit for predicting post-relapse survival of a multiple myeloma patient in a state of relapse, comprising the predictive model, as described supra, tangibly stored on a computer storage medium. Further to this embodiment, the kit comprises a platform having a plurality of probes hybridizable to a group of multiple myeloma genes consisting of, consisting essentially of, or comprising CCNE2, PECAM1, HMOX1, and CISH; optionally further comprising HBEGF, JUN, SIX5, and DUSP1; optionally further comprising BMP6, FOSB, and LIME1; optionally further comprising multiple myeloma genes BIRC3, FER1L4, KLHL21, MAFF, SOCS3, and TSC22D3. Further still, the kit may comprise a platform further having a plurality of probes hybridizable to one or more of multiple myeloma genes BIRC3, FER1L4, TSC22D3, MAFF, SOCS3 and KLHL21. In another related aspect, the probes are hybridizable to a group of multiple myeloma genes consisting of, consisting essentially of, or comprising multiple myeloma genes OSBPL10, CCNE2, UBE2D1, PECAM1, APOE, ATP6AP2, PTPRG, ZNF267, PECAM1, HMOX1, SOD2, TXNRD1, CD58, MS4A7, PPBP, EPAS1, FLNA, EPHB1, PLA2G7, CISH, SQSTM1, KLHL14, ATP6V1C1, FAM13A, PECAM1, SCD5, GM2A, CREB3L2, CD163, CPM, IDUA, SPAG4, and Clorf38. In yet another related aspect, the probes are hybridizable to a group of multiple myeloma genes consisting of, consisting essentially of, or comprising multiple myeloma genes AMPD1, ANPEP, BIRC3, BMP6, BTLA, CBFA2T3, CCNE2, CCR2, CCR7, CD44, CD48, CD82, CEP170, CHST11, CISH, CITED2, DUSP1, EPB41L3, FAM129B, FER1L4, FOS, FOSB, GLA, GPRC5D, HBEGF, HMOX1, ICAM1, IFI30, IL8, ISG20, JUN, KANK1, KLF2, KLHL21, LIME1, LRRN1, MAFF, MMP14, MT1E, MT1H, PECAM1, PLAC8, PLAU, PLAUR, PRRG4, SAT1, SGK1, SIX5, SLC25A19, SLC2A4RG, SOCS3SPHK1, TGM2, TMEM49, TNFAIP2, TNFRSF17, TSC22D3, and UGT2B1.

In yet another embodiment the present invention provides a method for identifying cancer genes predictive of post-relapse survival for a cancer patient, comprising co-culturing cancer cells with cells that interact with the cancer cells in their microenvironment; performing a first global gene expression profiling on the cancer cells before co-culture; performing a second global gene expression profiling on the cancer cells after co-culture; identifying via statistical analysis, from the first and second gene expression profile, a set of genes differentially expressed after co-culture; performing a third global gene expression profiling on post-relapse cancer cells obtained from relapsed cancer patients; identifying, via statistical analysis, from the third expression profile, those post-relapse genes that are also differentially expressed; and comparing post-relapse expression of these genes with duration of survival of the post-relapse cancer patients, thereby identifying the cancer genes predictive of post-relapse survival of the cancer patient. In one embodiment, the cancer cells are co-cultured with osteoclast cells. In another embodiment, the cancer cells are co-cultured with mesenchymal stem cells.

In a further embodiment the method may comprise performing a multivariate permutation test to eliminate genes with a higher than a pre-determined false positive rate of prediction. In another further embodiment the method may comprise identifying networks of interrelated genes among the differentially expressed gene set to further narrow the genes comprising the same. In all embodiments the ratio of change in expression may be a ratio of a change in signal intensity at relapse to a change in signal intensity at baseline. In an aspect of these embodiments the cancer cells may be multiple myeloma cells and the co-cultured cells are osteoclasts or mesenchymal stem cells.

Provided herein are methods, systems and kits utilizing a predictive model to predict post-survival relapse of a cancer patient, preferably, but not limited to, an individual with multiple myeloma. The predictive model is based on a group of genes that are shown statistically to beneficially affect the survival of myeloma cells after exposure to chemotherapeutic agents during a therapeutic regimen undergone by the patient. It is recognized that the global expression profiling techniques described herein are well-suited to identify genes associated with survival of other cancer cells and, as such, applicable predictive models can be constructed as predictive tools for calculating a risk of death for a cancer patient in which the cancer has relapsed. Platforms, such as DNA microarrays or RT-PCR arrays, measure gene expression levels and/or quantify signal intensity related to gene expression.

The predictive model provided herein is constructed utilizing gene expression values, i.e., levels, of the survival associated genes in relapse, such as the genes identified in Table 5 with the exception of PLAUR or, more preferably, the genes identified in Table 6, after the cancer patient has relapsed. A coefficient representing the weight contribution of each of the genes in promoting myeloma cell survival is determined based on the baseline gene expression values. This represents a sliding scale for assigning risk of death of the patient after relapse of the cancer. The predictive model comprises a calculator configured to utilize a summation function to calculate and assign risk. Risk is assigned based on the summation of products of the coefficient for each gene and the gene expression value of the gene after relapse.

The predictive model may be provided in a computer or other electronic device having one or more wired or wireless network connections, a memory to store the model and a processor to execute instructions enabling the predictive model on the computer or other electronic device. Such computers and electronic devices are well-known and standard in the art. The predictive model may comprise a computer program product tangibly stored in a memory on a computer or other computer storage device as are known in the art.

In constructing the predictive model provided herein, it is now widely accepted that the changes myeloma induces in the bone marrow microenvironment that result in osteolytic bone disease are not just manifestation. The cellular changes induced by myeloma cells supply factors and signals essential for the sustenance and progression of the disease. Co-cultures of myeloma cells with osteoclasts and with bone marrow mesenchymal stem cells are used to identify changes in gene expression by myeloma cells induced following these co-culture.

It is reasonable that genes required for myeloma cell survival will be among the genes whose expression similarly changes in both co-culture systems, such genes were selected for further study. It is interesting that from changes in the expression of over a thousand probe sets, only 72, corresponding to 58 genes, were common to both co-culture systems, indicating that the majority of the other observed changes were unique to the interaction of myeloma cells with osteoclasts or mesenchymal stem cells, and probably not associated with myeloma cell survival.

Changes in gene expression associated with myeloma cells survival are utilized as prognostic indicators. Expression of 22 of 58 genes changed in patients at relapse compared with baseline expression, and 7 genes (8 probesets) (PECAM1, ANPEP, PLAU (211668_S_AT), DUSP1, CCNE2, KLHL21, ICAM1 of these changes were significantly (p<0.05) associated with post relapse survival of myeloma cells. The genes associated with longer survival are:

1. Lower expression of the Wnt target regulator of cell cycle (CCNE2/Cyclin E2 (2);

2. Lower expression of KLHL21, which is a gene required for efficient chromosome alignment and cytokinesis (3);

3. Higher expression of the CD38 ligand PECAM1 (CD31), which is expressed on bone marrow myeloma cells, but not on extramedullary cells (4), targets cells for apoptosis and, together with cadherin 5 and β-catenin, is essential for angiogenesis (5);

4. Lower expression of ICAM-1 (CD54) whose expression is associated with cell adhesion mediated drug resistance (6) and is important for transendothelial migration (7);

5. Lower expression of PLAU 211668_S_AT (urokinase-type plasmin activator), a proteolytic enzyme that breaks down matrix and promotes invasion (8).

6. Higher expression of ANPEP (CD13) which is a protease present in soluble form in the plasma (9) and is involved in metabolism of regulatory peptides (10), is involved in tumor angiogenesis (111) and reduces availability of certain peptides to dendritic cells (9); and

7. Higher expression of DUSP1, the dual specificity phosphatase, a potential target of β-catenin that dephosphorylates Erks, JunK, and p38 MAPK and regulates the innate immune response (12).

While changes in gene expression at relapse point to emergence of more aggressive myeloma cells either by selection of pre-existing or new adaptations, they also mask the absolute level of expression, which by itself could be an important disease feature. Indeed, expression of 18 genes at relapse was significantly (p<0.05) associated with survival after relapse, some with high levels of significance.

In addition to the genes discussed above, other genes whose expression level is associated with longer survival of myeloma cells are:

8. Higher expression of components of the transcription regulator AP-1 JUN (13);

9. Higher expression of FOSB;

10. Higher expression of TSC22D3 (GILZ) which is a suppressor of AP-1 and NF-κB DNA binding activity (14);

11. Higher expression of HMOX1, the stress response heme oxigenase 1, which is upregulated in myeloma by oxidative stress (15-18); and

12. Lower expression of MAFF, a regulator of stress response and pro inflammatory cytokines, that is essential for antioxidant response element dependent genes and must cooperate with Nrf2 to elicit this response (19-21);

13. Higher expression of CISH, a member of the SOCS family which attenuate pro inflammatory signaling (22);

14. Higher expression of SIX5 which is expressed at low levels in many tissues, with known function in early development (23-26);

15. Higher expression of BMP6, known to inhibit proliferation of myeloma cell lines and survival of primary myeloma plasma cells and to confer better prognosis (227);

16. Lower expression of LIME1, the B-cell receptor and B-cell activator gene (28);

17. Higher expression of the inhibitor of apoptosis gene BIRC3 which is a cellular inhibitor of apoptosis 2, cIAP2, a target and regulator of NF-κB signaling, with lower expression in myeloma cells than normal plasma cells (29-30);

18. Higher expression of FER1L4 a trans membrane gene located to chromosome 20q11.23, whose function is as yet unknown; and

19. Higher expression of PLAUR(CD87), which was associated with better survival, in contrast with previous reports (8,31).

The strength of the association between the expression of these genes and post-relapse survival is evident by the ability of 18 of the genes (PLAUR the exception) to predict post relapse survival with a 4.4 hazard ratio. Furthermore, limiting the predictive model to those genes that have a false discovery rate of 5% or better, predicted post relapse survival of the 127 total therapy 2 patients with a hazard ratio of 8.4, of the 32 total therapy 3 patients with a hazard ratio of 5.6, and of the other 98 patients with a hazard ratio of 3.9.

While the association of higher expression of BMP6, CD31, or of the AP-1 complex and lower expression of CCNE2 makes mechanistic sense and higher expression of SIX5 could be signaling MMPC to differentiate, expression levels of other genes are unexpected. A higher expression of HMOX1 and of the inhibitor of apoptosis BIRC3 is associated with drug resistance and shorter survival, however, as demonstrated herein, higher expression of HMOX1 and BIRC3 correlated to an increase in survival of myeloma cells after treatment. In addition, SOCS3 and CISH are both suppressors of cytokine signaling and demonstrate opposite changes in expression levels. Furthermore, TSC22D3, an AP-1 suppressor gene, demonstrated higher expression.

Genes identified as increasing survival of cancer cells post treatment are potential therapeutic targets. Agents, such as chemotherapeutic agents, drugs or other compounds or biomolecules, effective to inhibit or prevent the increase or decrease of expression of the genes that confers post treatment survival to the cancer cells would improve therapeutic efficacy of a treatment regimen, decrease relapse and improve the cancer patient's chance for survival. Potential agents may be known in the art, may be synthesized or may be produced via standard molecular biological techniques. These agents may be tested in assays measuring gene expression levels and/or measuring gene products in cancer cell lines in vitro or in ex vivo samples in the presence or absence of chemotherapeutic agents utilized in known treatment regimens.

While the examples provided herein utilize multiple myeloma cells, one of ordinary skill in the art can see that the methods, systems and kits provided herein are readily adapted to any post-relapse situation. Global gene expression profiling and the statistical analysis techniques provided herein are well-suited to identify genes that are associated with the survival of cancer cells post treatment. The predictive model described herein can be configured for any cancer.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein Changes therein and other uses which are encompassed within the spirit of the invention as defined by the scope of the claims will occur to those skilled in the art.

### Example 1

### Methods and Materials

### Study Subjects

Gene expression profiles (GEP) of CD-138 selected myeloma cells were available on 127 patients with myeloma treated on total therapy 2 protocol (TT2) (32-22) at the time of first relapse (RL); for 71 of these patients, gene expression profiles was also analyzed prior to initiation of therapy (baseline, BL). These gene expression profiles data were used for post relapse survival analysis. Relapsed patients were treated with salvage therapy including thalidomide alone or in combination, lenalidomide alone or in combination, Bortezomib alone or in combination, BTD or BLD with or without chemotherapy (e.g. PACE), DT-PACE or VDT-PACE, or further transplant, as previously reported (34). Plasma cell purifications and gene expression profiles using the Affymetrix U133Plus2.0 microarray (Santa Clara, Calif.), were performed as previously described (35).

### Cells for Co-Culture Experiments

Multiple myeloma plasma cells (MMPC) were purified from heparinized bone marrow aspirates obtained from previously untreated patients with active MM during clinic visits, prior to initiation of treatment protocols. Multiple myeloma plasma cells were isolated using CD138 immunomagnetic bead selection and the automated autoMACs Separator (Miltenyi-Biotec, Auburn, Calif.). Multiple myeloma plasma cells purity was determined by CD38/CD45 flow cytometry to be routinely >95%.

Osteoclasts (OC) were prepared as previously described. Briefly, peripheral blood mononuclear cells (PBMC) were obtained from eight MM patients. Signed IRB-approved informed consent forms are kept on record. The cells were cultured at 2.5×10⁶ cells/ml in α-minimum essential medium (α-MEM) supplemented with 10% fetal bovine serum, antibiotics, RANKL (50 ng/ml), macrophage colony stimulating factor (M-CSF) (25 ng/ml), and 10 nM dexamethasone (Sigma, St. Louis, Mo.) (osteoclast media) for 10-14 days, at which time they contained large numbers of multinucleated, TRAP positive osteoclasts with bone-resorbing activity (36). RANKL and M-CSF were purchased from PeproTech, Princeton, N.J.

Mesenchymal cells from seven healthy donors were obtained from Darwin Prockop (Texas A & M Health Science Center College of Medicine Institute for Regenerative Medicine at Scott & White in Temple, Tex.). MSC were cultivated according to Dr. Prockop's established laboratory protocols (34).

### MMPC and OC Co-Cultures

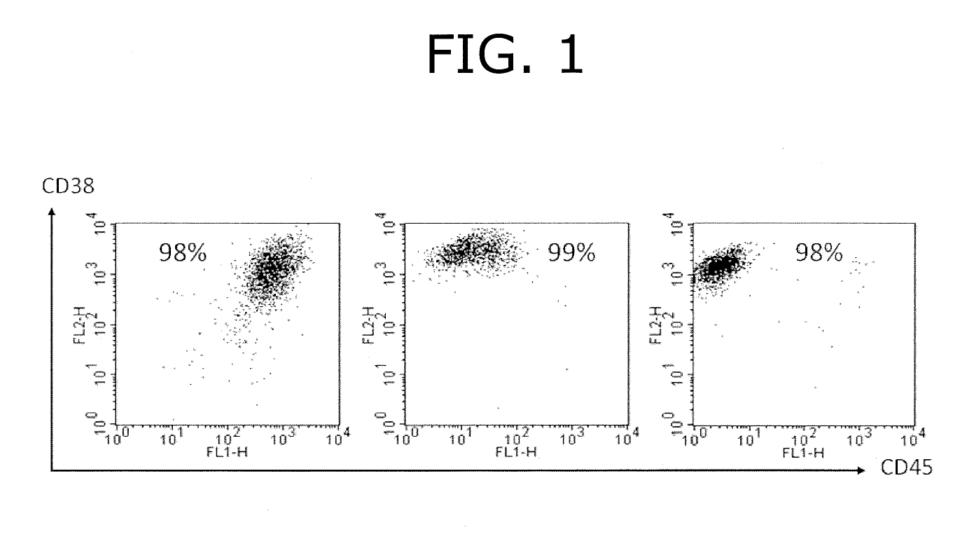

Osteoclast cultures were washed 3 times with phosphate-buffered saline to detach and remove any remaining non-adherent cells. For testing the molecular consequences of multiple myeloma plasma cells interaction with OC (MMPC/OC), 1.5×10⁶ CD138 sorted multiple myeloma plasma cells in 3 ml of osteoclast medium lacking dexamethasone were added per 30-mm diameter culture plates and the plates incubated for 4 days at 37° C. in a humidified atmosphere containing 5% CO₂. As reported previously, multiple myeloma plasma cells did not adhere to the osteoclasts and were easily recovered from co-cultures by gentle pipetting (33). The purity of recovered myeloma cells was evaluated by flow cytometry using PE-conjugated anti-CD38 and FITC-conjugated anti-CD45 monoclonal antibodies and was routinely ≧95% (FIG. 1). RNA was extracted using RNeasy kit (Qiagen) and DNA digested using RNase free DNase set (Qiagen) (35). RNA was similarly extracted from osteoclasts after co-culture, from OC cultured without multiple myeloma plasma cells, and from 0.5×10⁶ multiple myeloma plasma cells immediately after sorting and used as controls. Eight experiments were performed using multiple myeloma plasma cells from 8 patients and osteoclasts from 8 different patients.

### MMPC and MSC Co-Cultures

MSC were seeded in 24-well plates at 40,000 cells per well in complete culture medium at least 24 hours before adding multiple myeloma plasma cells, at which time the medium was removed and 1×10⁶ CD138-sorted (>95% viability as determined by trypan blue exclusion) multiple myeloma plasma cells in complete culture media were added to each well (MMPC/MSC). The plates were kept in a humidified atmosphere at 37° C. and 5% CO₂. After 18 hours incubation, the medium was carefully removed, total RNA extracted using RNeasy kit (Qiagen), and DNA digested using RNase free DNase set (Qiagen). For the control group, the medium was removed from MSC, and 1×10⁶ CD138-sorted (>95% viability) multiple myeloma plasma cells were added per well in phosphate-buffered saline in a total volume of ≦20 μL. Immediately afterward, the MSC+ multiple myeloma plasma cells mixture was lysed, and total RNA was extracted as described above.

### Example 2

### Analysis

### Analysis of Global Gene Expression

Global gene expression of multiple myeloma plasma cells/OC and MM/MSC interactions was analyzed using Affymetrix U133Plus2 chips. GeneChip Operating Software normalized output data (CHP files) were further analyzed using Acuity 4 bioinformatics software for analysis of microarrays (Molecular Devices, Sunnyvale, Calif.). To determine changes in gene expression, genes were selected that comply with the following three criteria: paired t-test p-value≦0.05, 500 mean signal cutoff in either pre- or post-co-culture, and at least a two-fold difference in mean signal as calculated by dividing the signal mean following co-culture by the signal mean before co-culture. Thereafter, the datasets selected for MMPC/MSC and MMPC/OC co-cultures were compared in order to identify genes whose expression was similarly changed in both co-culture systems. Ingenuity Pathways Analysis (IPA) software (Ingenuity Systems, Redwood City, Calif.) was used to identify networks of interrelated genes. IPA gene network score is the negative log of right-tailed Fisher's Exact Test p-value.

### Survival Analysis

To determine which, if any of the 58 genes, whose expression was changed in co-culture, were related to the clinical course of the disease, GEP of patients in relapse was analyzed using BRB-ArrayTools software (commercially available or available at www.linus.nci.nih.gov/BRB-ArrayTools.html) to identify genes associated with survival of these patients following relapse. A statistical significance level was computed for each gene, dichotomized at the median to low and high signal, based on univariate proportional hazards models (37). These p values were then used in a multivariate permutation test (38-39) in which the survival times and censoring indicators were randomly permutated among arrays. The multivariate permutation test was used to provide 90% confidence that the false discovery rate was less than 10%. The false discovery rate is the proportion of the list of genes claimed to be differentially expressed that are false positives.

To determine whether the extent of change of gene expression was related to outcome, among the 58 genes those were identified whose expression was also changed at relapse compared with baseline and calculated the ratio of change (signal at relapse/baseline signal were associated with post relapse survival. Survival graphs were generated using Kaplan-Meier methods, and the log-rank test was used for comparisons.

Analyses to Evaluate Possible Contamination of MMPC Cells after Coculture by OC

The purity of myeloma cells recovered from co-culture with osteoclasts (>95%) was the same as their purity at the start of the experiments, suggesting that the gene expression observed after co-culture are myeloma genes. Nevertheless, to ascertain that GEP of multiple myeloma plasma cells after co-culture does not represent contamination by osteoclasts or their progenitors, several analyses were performed.

In order to determine if genes expressed by myeloma cells after co-culture could reflect a small contamination of osteoclasts or their progenitors, probe sets were selected that were not expressed by myeloma cells prior to co-culture (detection p-value>0.05 and signal<500 in all 8 samples) and were highly expressed by osteoclasts after co-culture (detection p≦0.05 and signal range 3000-32587 in all 8 OC samples). 42 such probe sets were identified and for each the ratio of signals in multiple myeloma plasma cells after co-culture (signal range 98-32203) to the signals of OC from the same co-cultures was calculated. These ratios varied widely for each co-culture and between co-cultures, from a low of 0.03 to 2.94; there was no correlation between these ratios and osteoclast signal intensity. The median ratio for the 42 probesets across the 8 experiments was 0.4, range 0.1-1.0.

16 probesets were further selected that were expressed by OC after co-culture (mean signal 3008-7990) and multiple myeloma plasma cells prior to co-culture (mean signal 6366-28867). Expression of these probesets by multiple myeloma plasma cells after co-culture was reduced by 50 to 92%. There was no correlation between signal intensities by OC and reduction of expression (r=−0.10027), nor between the levels of expression by multiple myeloma plasma cells before co-culture and the Pre/Post ratios (r=−0.38973). These data clearly indicate that myeloma PC gene expression after co-culture with osteoclasts does not represent a small contamination by osteoclasts.

### Example 3

Multiple Myeloma Plasma Cells in Co-Culture with Osteoclasts

### Changes in Gene Expression by MMPC Following MMPC/OC Interaction.

Thirteen experiments using primary multiple myeloma plasma cells from eight patients and MSC from five healthy donors were carried out. Survival of multiple myeloma plasma cells in co-culture after 4-7 days was significantly higher (23% average) than controls (p<0.0002, 2-tailed Wilcoxon paired signed-rank test). Expression by myeloma cells of 887 Affymetrix probesets, representing 675 genes, was changed following interaction with osteoclasts (552 genes up regulated and 123 down regulated). Ingenuity Pathways Analysis software assigned 605 of these genes to 40 networks of interrelated genes, of them 33 with high IPA score in the range 8-42.

### Differentially Expressed Genes in MMPC/MSC Interaction

Following interaction of multiple myeloma plasma cells with MSC, expression of 365

Affymetrix probesets, corresponding to 296 genes (161 up regulated and 135 down regulated) was changed (Table 1). Ingenuity Pathways Analysis software assigned 244 of these 296 genes to 19 networks of interrelated genes, of them 16 with high IPA score in the range 12-41.

TABLE 1
Genes whose expression in myleoma cells changed
after co-cultue with mesenchymal stem cells (MSC)

| Affymetrix id | Gene Symbol | Chromosomal Location |
|---|---|---|
| 228038_at | SOX2 | chr3q26.3-q27 |
| 211506_s_at | IL8 | chr4q13-q21 |
| 204614_at | SERPINB2 | chr18q21.3 |
| 206336_at | CXCL6 | chr4q21 |
| 206337_at | CCR7 | chr17q12-q21.2 |
| 209277_at | TFPI2 | chr7q22 |
| 235638_at | RASSF6 | chr4q13.3 |
| 202859_x_at | IL8 | chr4q13-q21 |
| 219971_at | IL21R | chr16p11 |
| 221658_s_at | IL21R | chr16p11 |
| 223333_s_at | ANGPTL4 | chr19p13.3 |
| 209278_s_at | TFPI2 | chr7q22 |
| 223704_s_at | DMRT2 | chr9p24.3 |
| 1554997_a_at | PTGS2 | chr1q25.2-q25.3 |
| 221009_s_at | ANGPTL4 | chr19p13.3 |
| 209732_at | CLEC2B | chr12p13-p12 |
| 39402_at | IL1B | chr2q14 |
| 205266_at | LIF | chr22q12.2 |
| 202510_s_at | TNFAIP2 | chr14q32 |
| 213865_at | DCBLD2 | chr3q12.1\|3 |
| 211003_x_at | TGM2 | chr20q12 |
| 213506_at | F2RL1 | chr5q13 |
| 204748_at | PTGS2 | chr1q25.2-q25.3 |
| 227314_at | ITGA2 | chr5q23-q31 |
| 202638_s_at | ICAM1 | chr19p13.3-p13.2 |
| 238513_at | PRRG4 | chr11p13 |
| 210143_at | ANXA10 | chr4q33 |
| 203665_at | HMOX1 | chr22q12\|22q13.1 |
| 217165_x_at | MT1F | chr16q13 |
| 227697_at | SOCS3 | chr17q25.3 |
| 208581_x_at | MT1X | chr16q13 |
| 211573_x_at | TGM2 | chr20q12 |
| 202637_s_at | ICAM1 | chr19p13.3-p13.2 |
| 222068_s_at | LRRC50 | chr16q24.1 |
| 215485_s_at | ICAM1 | chr19p13.3-p13.2 |
| 213338_at | TMEM158 | chr3p21.3 |
| 215034_s_at | TM4SF1 | chr3q21-q25 |
| 204595_s_at | STC1 | chr8p21-p11.2 |
| 244721_at | TP53INP1 | chr8q22 |
| 206298_at | ARHGAP22 | chr10q11.22 |
| 205067_at | IL1B | chr2q14 |
| 201578_at | PODXL | chr7q32-q33 |
| 206432_at | HAS2 | chr8q24.12 |
| 206574_s_at | LOC100131062 /// | chr8q24.3 |
| | PTP4A3 | |
| 211668_s_at | PLAU | chr10q24 |
| 204338_s_at | RGS4 | chr1q23.3 |
| 205968_at | KCNS3 | chr2p24 |
| 204011_at | SPRY2 | chr13q31.1 |
| 204115_at | GNG11 | chr7q21 |
| 223986_x_at | DMRT2 | chr9p24.3 |
| 204597_x_at | STC1 | chr8p21-p11.2 |
| 221239_s_at | FCRL2 | chr1q21 |
| 208025_s_at | HMGA2 | chr12q15 |
| 201860_s_at | PLAT | chr8p12 |
| 230372_at | HAS2 | chr8q24.12 |
| 202888_s_at | ANPEP | chr15q25-q26 |
| 206461_x_at | MT1H | chr16q13 |
| 216336_x_at | MT1E /// MT1H /// | chr16q13 /// chr1q43 |
| | MT1M /// MT1P2 | |
| 219682_s_at | TBX3 | chr12q24.1 |
| 208937_s_at | ID1 | chr20q11 |
| 218451_at | CDCP1 | chr3p21.31 |
| 209621_s_at | PDLIM3 | chr4q35 |
| 205100_at | GFPT2 | chr5q34-q35 |
| 201650_at | JUP /// KRT19 | chr17q21 /// chr17q21.2 |
| 212185_x_at | MT2A | chr16q13 |
| 204745_x_at | MT1G | chr16q13 |
| 201625_s_at | INSIG1 | chr7q36 |
| 1560477_a_at | SAMD11 | chr1p36.33 |
| 210689_at | CLDN14 | chr21q22.3 |
| 211502_s_at | PFTK1 | chr7q21-q22 |
| 209387_s_at | TM4SF1 | chr3q21-q25 |
| 211924_s_at | PLAUR | chr19q13 |
| 218000_s_at | PHLDA1 | chr12q15 |
| 223222_at | SLC25A19 | chr17q25.3 |
| 213638_at | PHACTR1 | chr6p24.1 |
| 210538_s_at | BIRC3 | chr11q22 |
| 209695_at | LOC100131062 /// | chr8q24.3 |
| | PTP4A3 | |
| 209803_s_at | PHLDA2 | chr11p15.5 |
| 210916_s_at | CD44 | chr11p13 |
| 212859_x_at | MT1E | chr16q13 |
| 220994_s_at | STXBP6 | chr14q12 |
| 205032_at | ITGA2 | chr5q23-q31 |
| 201042_at | TGM2 | chr20q12 |
| 235548_at | APCDD1L | chr20q13.32 |
| 226462_at | STXBP6 | chr14q12 |
| 205924_at | RAB3B | chr1p32-p31 |
| 214430_at | GLA | chrXq22 |
| 201169_s_at | BHLHE40 | chr3p26 |
| 204339_s_at | RGS4 | chr1q23.3 |
| 219168_s_at | PRR5 | chr22q13 |
| 36711_at | MAFF | chr22q13.1 |
| 219134_at | ELTD1 | chr1p33-p32 |
| 221581_s_at | LAT2 | chr7q11.23 |
| 213256_at | MARCH3 | chr5q23.2 |
| 203180_at | ALDH1A3 | chr15q26.3 |
| 237411_at | ADAMTS6 | chr5q12 |
| 201920_at | SLC20A1 | chr2q11-q14 |
| 217279_x_at | MMP14 | chr14q11-q12 |
| 206953_s_at | LPHN2 | chr1p31.1 |
| 223101_s_at | ARPC5L | chr9q33.3 |
| 203068_at | KLHL21 | chr1p36.31 |
| 210845_s_at | PLAUR | chr19q13 |
| 226064_s_at | DGAT2 | chr11q13.5 |
| 211456_x_at | MT1P2 | chr1q43 |
| 223537_s_at | WNT5B | chr12p13.3 |
| 216250_s_at | LPXN | chr11q12.1 |
| 201976_s_at | MYO10 | chr5p15.1-p14.3 |
| 205207_at | IL6 | chr7p21 |
| 232122_s_at | VEPH1 | chr3q24-q25 |
| 224009_x_at | DHRS9 | chr2q31.1 |
| 212014_x_at | CD44 | chr11p13 |
| 221664_s_at | F11R | chr1q21.2-q21.3 |
| 203821_at | HBEGF | chr5q23 |
| 205034_at | CCNE2 | chr8q22.1 |
| 209386_at | TM4SF1 | chr3q21-q25 |
| 224911_s_at | DCBLD2 | chr3q12.1\|3 |
| 204490_s_at | CD44 | chr11p13 |
| 217127_at | CTH | chr1p31.1 |
| 228082_at | ASAM | chr11q24.1 |
| 210896_s_at | ASPH | chr8q12.1 |
| 226059_at | TOMM40L | chr1q23.3 |
| 212746_s_at | CEP170 | chr1q44 |
| 202748_at | GBP2 | chr1p22.2 |
| 1557905_s_at | CD44 | chr11p13 |
| 209835_x_at | CD44 | chr11p13 |
| 1555673_at | KAP2.1B /// | chr17q12-q21 /// |
| | KRTAP2-4 /// | chr17q21.2 |
| | LOC644350 /// | |
| | LOC728285 /// | |
| | LOC728934 /// | |
| | LOC730755 | |
| 203811_s_at | DNAJB4 | chr1p31.1 |
| 227340_s_at | RGMB | chr5q21.1 |
| 219634_at | CHST11 | chr12q |
| 232861_at | PDP2 | chr16q22.1 |
| 204445_s_at | ALOX5 | chr10q11.2 |
| 228703_at | P4HA3 | chr11q13.4 |
| 218368_s_at | TNFRSF12A | chr16p13.3 |
| 1554097_a_at | LOC554202 | chr9p21.3 |
| 201037_at | PFKP | chr10p15.3-p15.2 |
| 227123_at | RAB3B | chr1p32-p31 |
| 230799_at | LOC100134259 | chr2p21 |
| 1552717_s_at | CEP170 /// CEP170L | chr1q44 /// chr4q26 |
| 219257_s_at | SPHK1 | chr17q25.2 |
| 223019_at | FAM129B | chr9q34.11 |
| 204612_at | PKIA | chr8q21.12 |
| 204446_s_at | ALOX5 | chr10q11.2 |
| 209526_s_at | HDGFRP3 | chr15q25.2 |
| 202134_s_at | WWTR1 | chr3q23-q24 |
| 211343_s_at | COL13A1 | chr10q22 |
| 219985_at | HS3ST3A1 | chr17p12-p11.2 |
| 217999_s_at | PHLDA1 | chr12q15 |
| 224097_s_at | F11R | chr1q21.2-q21.3 |
| 206584_at | LY96 | chr8q21.11 |
| 214577_at | MAP1B | chr5q13 |
| 204224_s_at | GCH1 | chr14q22.1-q22.2 |
| 230290_at | SCUBE3 | chr6p21.3 |
| 201629_s_at | ACP1 | chr2p25 |
| 204604_at | PFTK1 | chr7q21-q22 |
| 223618_at | FMN2 | chr1q43 |
| 204749_at | NAP1L3 | chrXq21.3-q22 |
| 223952_x_at | DHRS9 | chr2q31.1 |
| 212003_at | C1orf144 | chr1p36.13 |
| 237563_s_at | LOC440731 | chr1q42.2 |
| 228776_at | GJC1 | chr17q21.31 |
| 213988_s_at | SAT1 | chrXp22.1 |
| 228266_s_at | HDGFRP3 | chr15q25.2 |
| 217998_at | LOC652993 /// PHLDA1 | chr12q15 /// chr12q21 |
| 201739_at | SGK1 | chr6q23 |
| 205479_s_at | PLAU | chr10q24 |
| 228415_at | AP1S2 | chrXp22.2 |
| 209524_at | HDGFRP3 | chr15q25.2 |
| 228253_at | LOXL3 | chr2p13 |
| 203904_x_at | CD82 | chr11p11.2 |
| 227405_s_at | FZD8 | chr10p11.21 |
| 209897_s_at | SLIT2 | chr4p15.2 |
| 205428_s_at | CALB2 | chr16q22.2 |
| 210513_s_at | VEGFA | chr6p12 |
| 219926_at | POPDC3 | chr6q21 |
| 223249_at | CLDN12 | chr7q21 |
| 230792_at | FAAH2 | chrXp11.1 |
| 206571_s_at | MAP4K4 | chr2q11.2-q12 |
| 206085_s_at | CTH | chr1p31.1 |
| 202132_at | WWTR1 | chr3q23-q24 |
| 209032_s_at | CADM1 | chr11q23.2 |
| 203939_at | NT5E | chr6q14-q21 |
| 209676_at | TFPI | chr2q32 |
| 238542_at | ULBP2 | chr6q25 |
| 223961_s_at | CISH | chr3p21.3 |
| 213310_at | EIF2C2 | chr8q24 |
| 212662_at | PVR | chr19q13.2 |
| 224774_s_at | NAV1 | chr1q32.3 |
| 225827_at | EIF2C2 | chr8q24 |
| 47069_at | PRR5 | chr22q13 |
| 223614_at | MMP16 | chr8q21.3 |
| 1555638_a_at | SAMSN1 | chr21q11 |
| 221845_s_at | CLPB | chr11q13.4 |
| 222548_s_at | MAP4K4 | chr2q11.2-q12 |
| 1569003_at | TMEM49 | chr17q23.1 |
| 216693_x_at | HDGFRP3 | chr15q25.2 |
| 206710_s_at | EPB41L3 | chr18p11.32 |
| 220161_s_at | EPB41L4B | chr9q31-q32 |
| 201422_at | IFI30 | chr19p13.1 |
| 208708_x_at | EIF5 | chr14q32.32 |
| 219878_s_at | KLF13 | chr15q12 |
| 1562460_at | CNDP2 | chr18q22.3 |
| 1553995_a_at | NT5E | chr6q14-q21 |
| 236656_s_at | LOC100130506 | — |
| 204220_at | GMFG | chr19q13.2 |
| 202869_at | OAS1 | chr12q24.1 |
| 210655_s_at | FOXO3 /// ZNF286C | chr17p11.2 /// chr6q21 |
| 221527_s_at | PARD3 | chr10p11.22-p11.21 |
| 206809_s_at | HNRNPA3 /// | chr10q11.21 /// chr2q31.2 |
| | HNRNPA3P1 | |
| 205469_s_at | IRF5 | chr7q32 |
| 235010_at | LOC729013 | chr11p15.3 |
| 230128_at | IGL@ | chr22q11.1-q11.2 |
| 222621_at | DNAJC1 | chr10p12.31 |
| 226659_at | DEF6 | chr6p21.33-p21.1 |
| 228291_s_at | NCRNA00153 | chr20pter-q11.23 |
| 213521_at | PTPN18 | chr2q21.1 |
| 209282_at | PRKD2 | chr19q13.3 |
| 218747_s_at | TAPBPL | chr12p13.31 |
| 206641_at | TNFRSF17 | chr16p13.1 |
| 204821_at | BTN3A3 | chr6p21.3 |
| 214209_s_at | ABCB9 | chr12q24 |
| 203615_x_at | SULT1A1 | chr16p12.1 |
| 225941_at | EIF4E3 | chr3p14 |
| 225728_at | SORBS2 | chr4q35.1 |
| 202265_at | BMI1 | chr10p11.23 |
| 220998_s_at | UNC93B1 | chr11q13 |
| 213540_at | HSD17B8 | chr6p21.3 |
| 212093_s_at | MTUS1 | chr8p22 |
| 236436_at | SLC25A45 | chr11q13.1 |
| 239412_at | IRF5 | chr7q32 |
| 209291_at | ID4 | chr6p22-p21 |
| 206170_at | ADRB2 | chr5q31-q32 |
| 216981_x_at | SPN | chr16p11.2 |
| 230388_s_at | LOC644246 | chr17q21.31 |
| 227087_at | INPP4A | chr2q11.2 |
| 209357_at | CITED2 | chr6q23.3 |
| 222762_x_at | LIMD1 | chr3p21.3 |
| 203837_at | MAP3K5 | chr6q22.33 |
| 219441_s_at | LRRK1 | chr15q26.3 |
| 221989_at | RPL10 | chrXq28 |
| 226104_at | RNF170 | chr8p11.21 |
| 227817_at | PRKCB | chr16p11.2 |
| 225981_at | C17orf28 | chr17q25.1 |
| 226452_at | PDK1 | chr2q31.1 |
| 242414_at | QPRT | chr16p11.2 |
| 235475_at | LOC100129720 | chr3q25.1 |
| 219541_at | LIME1 | chr20q13.3 |
| 225136_at | PLEKHA2 | chr8p11.23 |
| 201041_s_at | DUSP1 | chr5q34 |
| 205504_at | BTK | chrXq21.33-q22 |
| 215299_x_at | SULT1A1 | chr16p12.1 |
| 209230_s_at | NUPR1 | chr16p11.2 |
| 203317_at | PSD4 | chr2q13 |
| 204118_at | CD48 | chr1q21.3-q22 |
| 227134_at | SYTL1 | chr1p36.11 |
| 229009_at | SIX5 | chr19q13.32 |
| 202075_s_at | PLTP | chr20q12-q13.1 |
| 225763_at | RCSD1 | chr1q22-q24 |
| 203408_s_at | SATB1 | chr3p23 |
| 207777_s_at | SP140 | chr2q37.1 |
| 219865_at | HSPC157 /// | chr1p36.12 |
| | LOC100128919 | |
| 204552_at | INPP4A | chr2q11.2 |
| 207957_s_at | PRKCB | chr16p11.2 |
| 218058_at | CXXC1 | chr18q12 |
| 227429_at | EFCAB4A | chr11p15.5 |
| 217192_s_at | PRDM1 | chr6q21-q22.1 |
| 231647_s_at | FCRL5 | chr1q21 |
| 208983_s_at | PECAM1 | chr17q23 |
| 220565_at | CCR10 | chr17q21.1-q21.3 |
| 206589_at | GFI1 | chr1p22 |
| 225721_at | SYNPO2 | chr4q26 |
| 226489_at | TMCC3 | chr12q22 |
| 226132_s_at | MANEAL | chr1p34.3 |
| 223569_at | PPAPDC1B | chr8p12 |
| 33304_at | ISG20 | chr15q26 |
| 203153_at | IFIT1 | chr10q25-q26 |
| 219143_s_at | RPP25 | chr15q24.1 |
| 212096_s_at | MTUS1 | chr8p22 |
| 232213_at | PELI1 | chr2p13.3 |
| 206176_at | BMP6 | chr6p24-p23 |
| 1559584_a_at | C16orf54 /// | chr16p11.2 |
| | hCG_1644884 | |
| 218437_s_at | LZTFL1 | chr3p21.3 |
| 223358_s_at | PDE7A | chr8q13 |
| 228897_at | DERL3 | chr22q11.23 |
| 229390_at | FAM26F | chr6q22.1 |
| 221727_at | SUB1 | chr5p13.3 |
| 219961_s_at | NCRNA00153 | chr20pter-q11.23 |
| 229497_at | ANKDD1A | chr15q22.31 |
| 209827_s_at | IL16 | chr15q26.3 |
| 218494_s_at | SLC2A4RG | chr20q13.33 |
| 208981_at | PECAM1 | chr17q23 |
| 215923_s_at | PSD4 | chr2q13 |
| 213005_s_at | KANK1 | chr9p24.3 |
| 236226_at | BTLA | chr3q13.2 |
| 219014_at | PLAC8 | chr4q21.22 |
| 226140_s_at | OTUD1 | chr10p12.2 |
| 227074_at | LOC100131564 | chr1p22.1 |
| 224724_at | SULF2 | chr20q12-q13.2 |
| 208982_at | PECAM1 | chr17q23 |
| 229530_at | GUCY1A3 | chr4q31.3-q33\|4q31.1-q31.2 |
| 208056_s_at | CBFA2T3 | chr16q24 |
| 223044_at | SLC40A1 | chr2q32 |
| 205671_s_at | HLA-DOB | chr6p21.3 |
| 206150_at | CD27 | chr12p13 |
| 224722_at | MIB1 | chr18q11.2 |
| 204698_at | ISG20 | chr15q26 |
| 207001_x_at | TSC22D3 | chrXq22.3 |
| 203111_s_at | PTK2B | chr8p21.1 |
| 219371_s_at | KLF2 | chr19p13.13-p13.11 |
| 226384_at | PPAPDC1B | chr8p12 |
| 218048_at | COMMD3 | chr10pter-q22.1 |
| 222245_s_at | FER1L4 | chr20q11.22 |
| 201465_s_at | JUN | chr1p32-p31 |
| 203836_s_at | MAP3K5 | chr6q22.33 |
| 227865_at | C9orf103 | chr9q21-q22 |
| 226646_at | KLF2 | chr19p13.13-p13.11 |
| 203110_at | PTK2B | chr8p21.1 |
| 212225_at | EIF1 | chr17q21.2 |
| 235401_s_at | FCRLA | chr1q23.3 |
| 218409_s_at | DNAJC1 | chr10p12.31 |
| 227711_at | GTSF1 | chr12q13.2 |
| 207794_at | CCR2 /// FLJ78302 | chr3p21.31 |
| 201694_s_at | EGR1 | chr5q31.1 |
| 51158_at | FAM174B | chr15q26.1 |
| 225579_at | PQLC3 | chr2p25.1 |
| 225895_at | SYNPO2 | chr4q26 |
| 226558_at | LOC653071 | — |
| 219525_at | SLC47A1 | chr17p11.2 |
| 210889_s_at | FCGR2B | chr1q23 |
| 213160_at | DOCK2 | chr5q35.1 |
| 227404_s_at | EGR1 | chr5q31.1 |
| 227641_at | FBXL16 | chr16p13.3 |
| 205804_s_at | TRAF3IP3 | chr1q32.3-q41 |
| 206478_at | KIAA0125 | chr14q32.33 |
| 207245_at | UGT2B17 | chr4q13 |
| 226344_at | ZMAT1 | chrXq21 |
| 221666_s_at | PYCARD | chr16p12-p11.2 |
| 205901_at | PNOC | chr8p21 |
| 207980_s_at | CITED2 | chr6q23.3 |
| 207321_s_at | ABCB9 | chr12q24 |
| 201466_s_at | JUN | chr1p32-p31 |
| 233555_s_at | SULF2 | chr20q12-q13.2 |
| 204794_at | DUSP2 | chr2q11 |
| 205774_at | F12 | chr5q33-qter |
| 1554208_at | MEI1 | chr22q13.2 |
| 204192_at | CD37 | chr19q13.3 |
| 227235_at | GUCY1A3 | chr4q31.3-q33\|4q31.1-q31.2 |
| 213888_s_at | LOC100133233 /// | chr1q32.2 /// chr1q32.3-q41 |
| | TRAF3IP3 | |
| 1568964_x_at | SPN | chr16p11.2 |
| 221297_at | GPRC5D | chr12p13.3 |
| 225792_at | HOOK1 | chr1p32.1 |
| 206687_s_at | PTPN6 | chr12p13 |
| 207996_s_at | C18orf1 | chr18p11.2 |
| 225720_at | SYNPO2 | chr4q26 |
| 206978_at | CCR2 /// FLJ78302 | chr3p21.31 |
| 218918_at | MAN1C1 | chr1p35 |
| 232304_at | PELI1 | chr2p13.3 |
| 201044_x_at | DUSP1 | chr5q34 |
| 211998_at | H3F3A /// H3F3B /// | chr17q25 /// chr1q41 /// |
| | LOC440926 | chr2q31.1 |
| 226884_at | LRRN1 | chr3p26.2 |
| 206121_at | AMPD1 | chr1p13 |
| 230011_at | MEI1 | chr22q13.2 |
| 202340_x_at | NR4A1 | chr12q13 |
| 220377_at | FAM30A | chr14q32.33 |
| 209189_at | FOS | chr14q24.3 |
| 202768_at | FOSB | chr19q13.32 |

### Differentially Expressed Genes by MMPC Common to Both Co-Culture Systems:

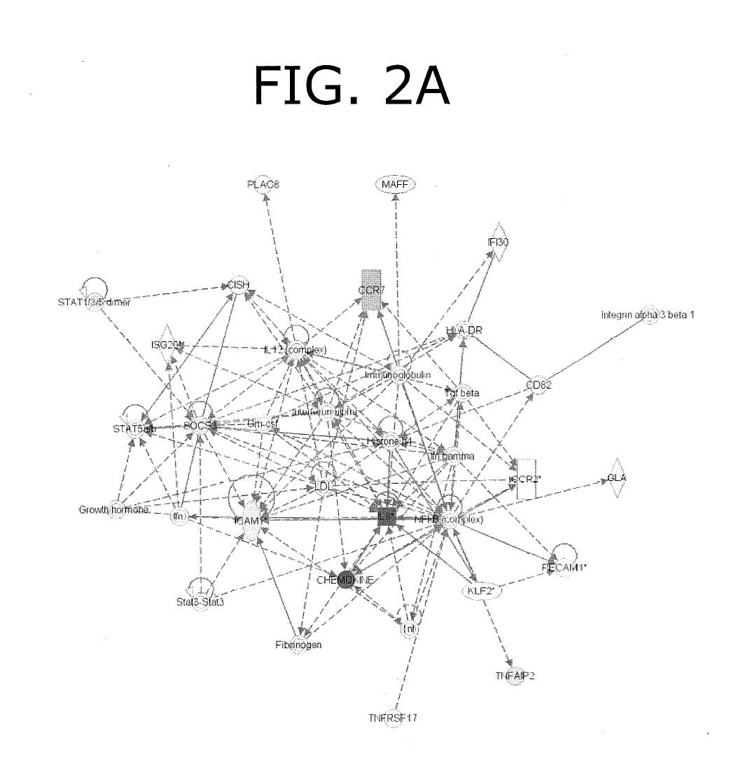

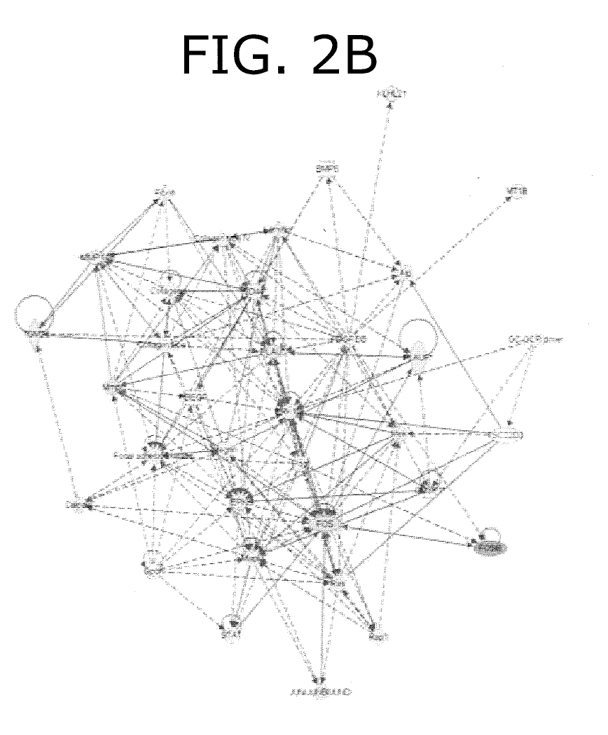

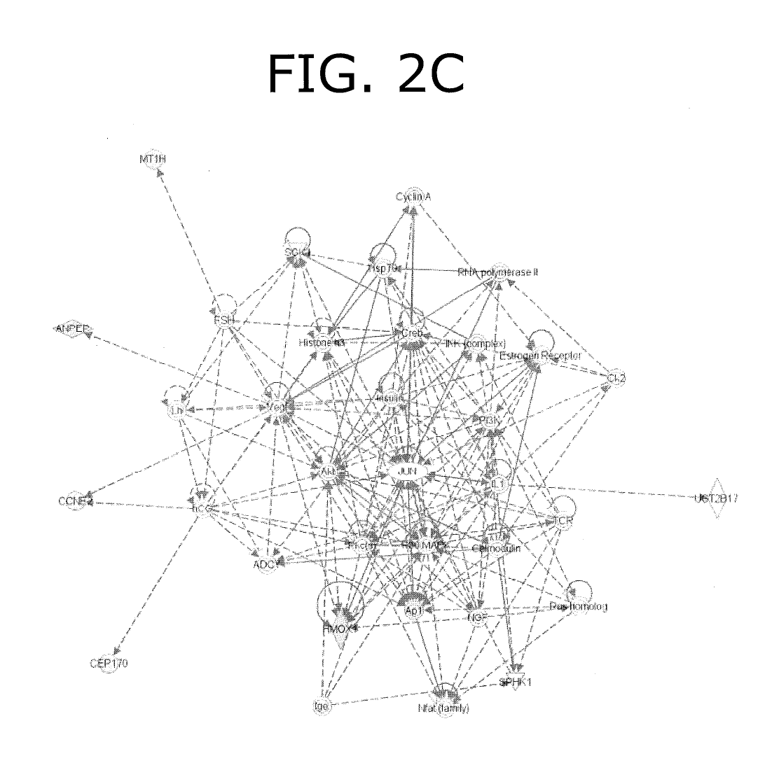

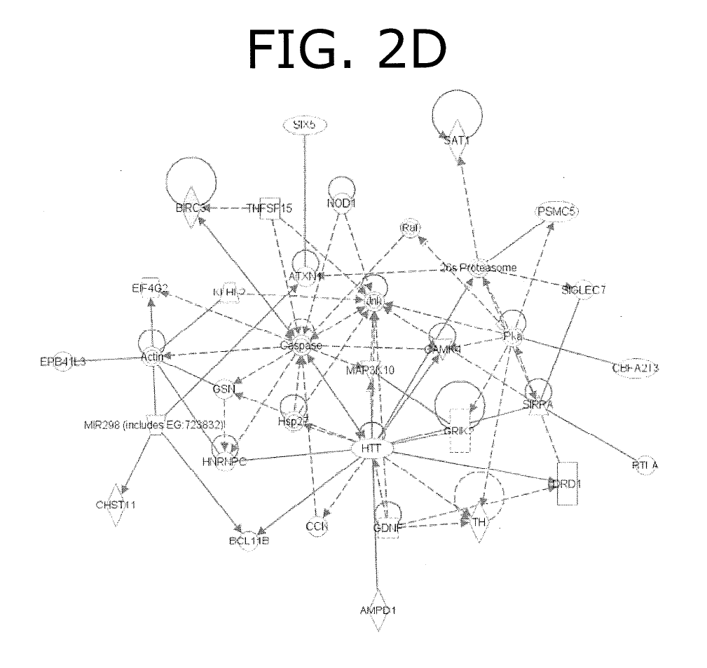

Comparison of genes whose expression was changed in multiple myeloma plasma cells following co-culture with osteoclasts and genes whose expression was similarly changed in MMPC/MSC co-culture identified 72 commonly changed probesets, representing 58 genes; 33 genes were up regulated and 25 down regulated. The 58 genes include one cytokine, 12 transcription regulators, two growth factors, 16 enzymes, five receptors, one transporter and 22 with other functions (Table 2). Using IPA, 54 of the 58 genes (72 probesets) were assigned to five distinguished networks on interrelated genes with high probability IPA scores (FIGS. 2A-2E).

TABLE 2
72 probe sets whose expression was similarly altered in MMPC
following co-culture with osteoclasts and osteoblasts

| | | | Ratio of means | Ratio of means |
|---|---|---|---|---|
| | | | MM/OC | MM/MSC |
| | | | after/before | after/before |
| Type | Affymetrix ID | Symbol | interaction | interaction |
| Cytokine | 202859_x_at | IL8 | 50 | 5.56 |
| | 211506_s_at | IL8 | 50 | 11.11 |
| Phosphatase | 201044_x_at | DUSP1 | 0.26 | 0.24 |
| | 201041_s_at | DUSP1 | 0.08 | 0.48 |
| Peptidases | 202888_s_at | ANPEP | 14.29 | 2.7 |
| | 217279_x_at | MMP14 | 4 | 2.27 |
| | 205479_s_at | PLAU | 7.69 | 1.92 |
| | 211668_s_at | PLAU | 9.09 | 2.94 |
| Other enzymes | 206121_at | AMPD1 | 0.21 | 0.23 |
| | 207245_at | UGT2B17 | 0.21 | 0.33 |
| | 204698_at | ISG20 | 0.43 | 0.39 |
| | 33304_at | ISG20 | 0.44 | 0.43 |
| | 201422_at | IFI30 | 5.88 | 1.82 |
| | 213988_s_at | SAT1 | 3.03 | 1.96 |
| | 219634_at | CHST11 | 2.04 | 2.08 |
| | 214430_at | GLA | 2.86 | 2.38 |
| | 201042_at | TGM2 | 20 | 2.44 |
| | 210538_s_at | BIRC3 | 2.5 | 2.5 |
| | 203665_at | HMOX1 | 9.09 | 3.33 |
| G-protein | 206978_at | CCR2 | 0.31 | 0.26 |
| coupled | 207794_at | CCR2 | 0.28 | 0.37 |
| receptors | 206337_at | CCR7 | 2.13 | 6.25 |
| | 221297_at | GPRC5D | 0.31 | 0.28 |
| Growth factors | 206176_at | BMP6 | 0.36 | 0.43 |
| | 203821_at | HBEGF | 3.45 | 2.17 |
| Kinase | 201739_at | SGK1 | 7.14 | 1.96 |
| | 219257_s_at | SPHK1 | 2.38 | 2.04 |
| Transcription | 202768_at | FOSB | 0.05 | 0.03 |
| regulators | 209189_at | FOS | 0.07 | 0.07 |
| | 207980_s_at | CITED2 | 0.3 | 0.33 |
| | 226646_at | KLF2 | 0.43 | 0.37 |
| | 201465_s_at | JUN | 0.27 | 0.38 |
| | 219371_s_at | KLF2 | 0.26 | 0.39 |
| | 207001_x_at | TSC22D3 | 0.19 | 0.39 |
| | 208056_s_at | CBFA2T3 | 0.47 | 0.4 |
| | 213005_s_at | KANK1 | 0.45 | 0.41 |
| | 218494_s_at | SLC2A4RG | 0.45 | 0.42 |
| | 229009_at | SIX5 | 0.28 | 0.47 |
| | 209357_at | CITED2 | 0.42 | 0.49 |
| | 36711_at | MAFF | 2.56 | 2.33 |
| Transmembrane | 210845_s_at | PLAUR | 11.11 | 2.22 |
| receptors | 211924_s_at | PLAUR | 8.33 | 2.56 |
| | 215485_s_at | ICAM1 | 3.33 | 3.13 |
| | 202637_s_at | ICAM1 | 3.85 | 3.23 |
| | 202638_s_at | ICAM1 | 4.55 | 3.45 |
| | 1557905_s_at | CD44 | 3.85 | 2.08 |
| | 209835_x_at | CD44 | 4.55 | 2.08 |
| | 212014_x_at | CD44 | 5 | 2.17 |
| | 208982_at | PECAM1 | 0.45 | 0.4 |
| | 208981_at | PECAM1 | 0.47 | 0.41 |
| | 208983_s_at | PECAM1 | 0.39 | 0.45 |
| Transporter | 223222_at | SLC25A19 | 3.03 | 2.5 |
| Other | 236226_at | BTLA | 0.48 | 0.41 |
| | 205034_at | CCNE2 | 3.23 | 2.17 |
| | 204118_at | CD48 | 0.35 | 0.47 |
| | 203904_x_at | CD82 | 3.7 | 1.92 |
| | 212746_s_at | CEP170 | 4.76 | 2.13 |
| | 223961_s_at | CISH | 2.78 | 1.85 |
| | 206710_s_at | EPB41L3 | 33.33 | 1.82 |
| | 223019_at | FAM129B | 6.25 | 2.04 |
| | 222245_s_at | FER1L4 | 0.42 | 0.38 |
| | 203068_at | KLHL21 | 2.94 | 2.27 |
| | 219541_at | LIME1 | 0.25 | 0.48 |
| | 226884_at | LRRN1 | 0.31 | 0.23 |
| | 212859_x_at | MT1E | 2.5 | 2.44 |
| | 206461_x_at | MT1H | 2.94 | 2.7 |
| | 219014_at | PLAC8 | 0.47 | 0.41 |
| | 238513_at | PRRG4 | 2.63 | 3.33 |
| | 227697_at | SOCS3 | 3.45 | 3.23 |
| | 1569003_at | TMEM49 | 4.17 | 1.85 |
| | 202510_s_at | TNFAIP2 | 4.76 | 3.7 |
| | 206641_at | TNFRSF17 | 0.43 | 0.5 |

To investigate if these 58 genes are relevant to the biology of myeloma as expressed by correlation with the clinical course of the disease, those genes whose expression by myeloma cells obtained at relapse is significantly associated with post relapse survival were identified. Of the 58 genes, 22 genes (27 probesets, Table 3) changed expression after relapse compared with baseline in the 71 relapsed patients treated on TT2 for whom baseline and relapse GEP were available. The change in expression of these 72 probesets was calculated as the ratios of signal at relapse/baseline signal. Ratios of 8 probesets, representing 7 genes, dichotomized at the median, were significantly associated with survival at 0.05 level of univariate analysis. These probesets are listed in Table 4 in order of the univariate test p-value.

TABLE 3
27 probesets whose expression changed following
co-culture also changed at relapse*

| | | Baseline | Relapse | p | Ratio RL/BL |
|---|---|---|---|---|---|
| | Gene | Median | Median | (Paired | Median |
| Probe set | Symbol | (range) | (range) | t-Test) | (range) |
| 208056_s_at | CBFA2T3 | 1479 | 1285 | <0.001 | 0.96 |
| | | (444-4510) | (251-2824) | | (0.03-5.4) |
| 207980_s_at | CITED2 | 6341 | 5268 | 0.001 | 0.89 |
| | | (415-31118) | (277-22292) | | (0.008-1.80) |
| 209357_at | CITED2 | 8893 | 7636 | 0.009 | 0.71 |
| | | (93-22490) | (12-23638) | | (0.04-5.87) |
| 201041_s_at | DUSP1 | 24359 | 22540 | 0.049 | 0.83 |
| | | (1892-42774) | (1075-48866) | | (0.26-1.88) |
| 221297_at | GPRC5D | 7488 | 6228 | 0.029 | 0.63 |
| | | (709-27040) | (504-29988) | | (0.02-2.15) |
| 213005_s_at | KANK1 | 6885 | 4470 | 0.000 | 0.86 |
| | | (900-17834) | (650-27206) | | (0.04-2.08) |
| 226646_at | KLF2 | 852 | 736 | 0.001 | 0.91 |
| | | (193-3233) | (134-2376) | | (0.02-6.20) |
| 208981_at | PECAM1 | 9833 | 6547 | 0.000 | 0.80 |
| | | (323-19790) | (1343-15633) | | (0.001-3.51) |
| 208982_at | PECAM1 | 9785 | 8288 | 0.005 | 0.73 |
| | | (535-41487) | (285-36351) | | (0.05-5.33) |
| 208983_s_at | PECAM1 | 4679 | 3412 | 0.023 | 0.88 |
| | | (174-15539) | (18-11671) | | (0.10-5.71) |
| 206641_at | TNFRSF17 | 20293 | 18747 | 0.037 | 0.90 |
| | | (6980-37358) | (166-32405) | | (0.19-2.49) |
| 203665_at | HMOX1 | 326 | 780 | 0.000 | 1.32 |
| | | (25-2747) | (83-18250) | | (0.18-11.44) |
| 203821_at | HBEGF | 106.906 | 158 | 0.004 | 1.17 |
| | | (21-815) | (21-4625) | | (0.11-13.93) |
| 206710_s_at | EPB41L3 | 45.167 | 82 | 0.010 | 0.69 |
| | | (3-414) | (5-1604) | | (0.10-5.4) |
| 203068_at | KLHL21 | 111.004 | 193 | 0.000 | 1.48 |
| | | (43-1005) | (14-1427) | | (0.14-15.54) |
| 201422_at | IFI30 | 1381 | 1780 | 0.001 | 2.22 |
| | | (102-7766) | (317-18411) | | (0.09-88.54) |
| 215485_s_at | ICAM1 | 181 | 297 | 0.003 | 1.62 |
| | | (28-1172) | (28-1384) | | (0.17-30.45) |
| 223222_at | SLC25A19 | 465 | 627 | 0.000 | 1.30 |
| | | (32-1550) | (219-1521) | | (0.28-7.35) |
| 219634_at | CHST11 | 780 | 1072 | 0.001 | 0.68 |
| | | (166-2131) | (65-4702) | | (0.05-8.12) |
| 205479_s_at | PLAU | 74 | 97 | 0.016 | 1.63 |
| | | (23-732) | (26-869) | | (0.02-21.17) |
| 202638_s_at | ICAM1 | 448 | 573 | 0.048 | 0.79 |
| | | (113-2607) | (55-4685) | | (0.06-14.55) |
| 214430_at | GLA | 756 | 833 | 0.008 | 0.74 |
| | | (259-2904) | (401-4876) | | (0.05-4.94) |
| 223961_s_at | CISH | 316 | 266 | 0.03 | 0.89 |
| | | (19-3165) | (10-1127) | | (0-1.8) |
| 202859_x_at | IL8 | 319 | 223 | 0.047 | 1.48 |
| | | (59-1672) | (41-1266) | | (0.13-15.53) |
| 211506_s_at | IL8 | 83 | 62 | 0.03 | 2.22 |
| | | (6-684) | (5-294) | | (0.09-88.54) |
| 206461_x_at | MT1H | 216 | 149 | 0.049 | 1.63 |
| | | (32-1013) | (27-1660) | | (0.01-21.17) |
| 211924_s_at | PLAUR | 305 | 179 | 0.01 | 1.14 |
| | | (50-1421) | (9-1179) | | (0.17-6.33) |

*Of the 72 probesets whose expression by myeloma plasma cells was altered after co-culture, expression of 27 was also changed at relapse compared with baseline.

TABLE 4
Genes whose change in expression correlated with post-relapse survival.

| | Gene | Parametric | Permutation | Hazard | Signal Ratio^{&} | |
|---|---|---|---|---|---|---|
| Probe set | symbol | p-value | p-value* | Ratio^{#} | median (range) | |
| 205034_at | CCNE2 | 0.017 | 0.0163 | 1.6 | 1.04 | (0.27-7.69) |
| 208983_s_at | PECAM1 | 0.031 | 0.0307 | 0.45 | 0.92 | (0.016-6.2) |
| 203068_at | KLHL21 | 0.032 | 0.0342 | 1.321 | 1.54 | (0.06-15.54) |
| 202638_s_at | ICAM1 | 0.038 | 0.0428 | 1.546 | 1.2 | (0.11-13.9) |
| 211668_s_at | PLAU | 0.039 | 0.0384 | 1.377 | 1 | (0.05-13.67) |
| 202888_s_at | ANPEP | 0.042 | 0.044 | 0.591 | 0.92 | (0.49-10.23) |
| 205479_s_at | PLAU | 0.043 | 0.045 | 0.631 | 1.2 | (0.28-7.35) |
| 201044_x_at | DUSP1 | 0.049 | 0.051 | 0.68 | 1.22 | (0.007-15.73) |

*Permutation p-values for significant genes were computed based on 10000 random permutations.
^{#}Hazard ratio is the ratio of hazards for a two-fold change in the gene expression level.
^{&}Ratio was calculated as signal at relapse/baseline signal.

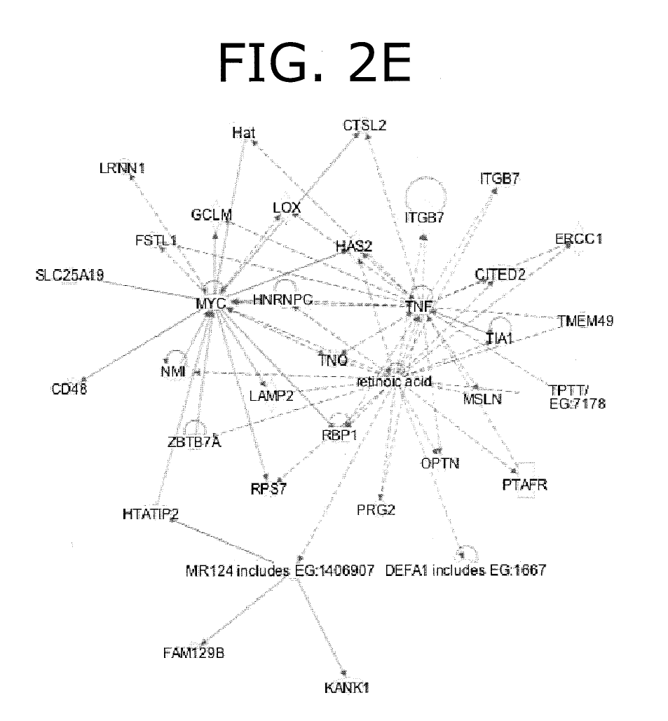

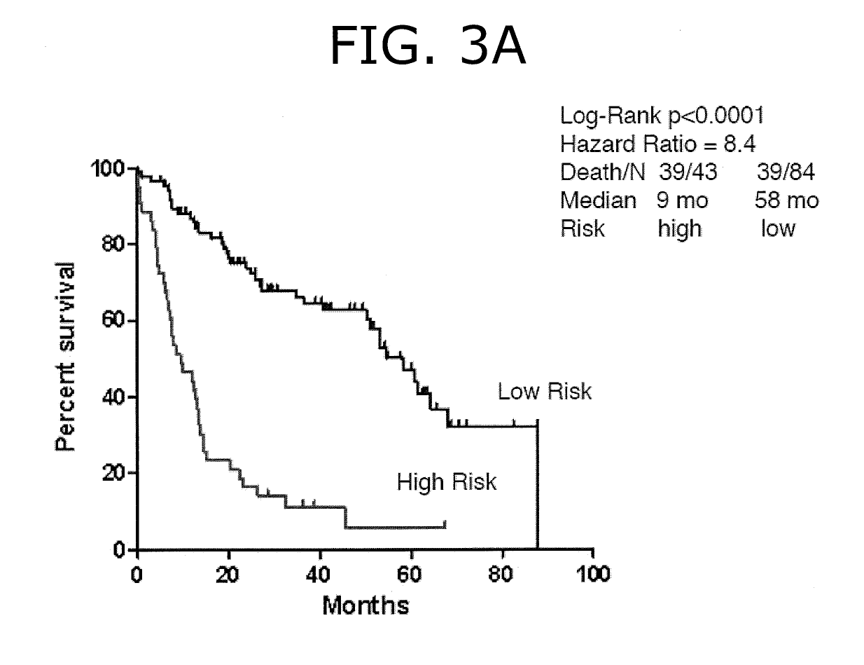

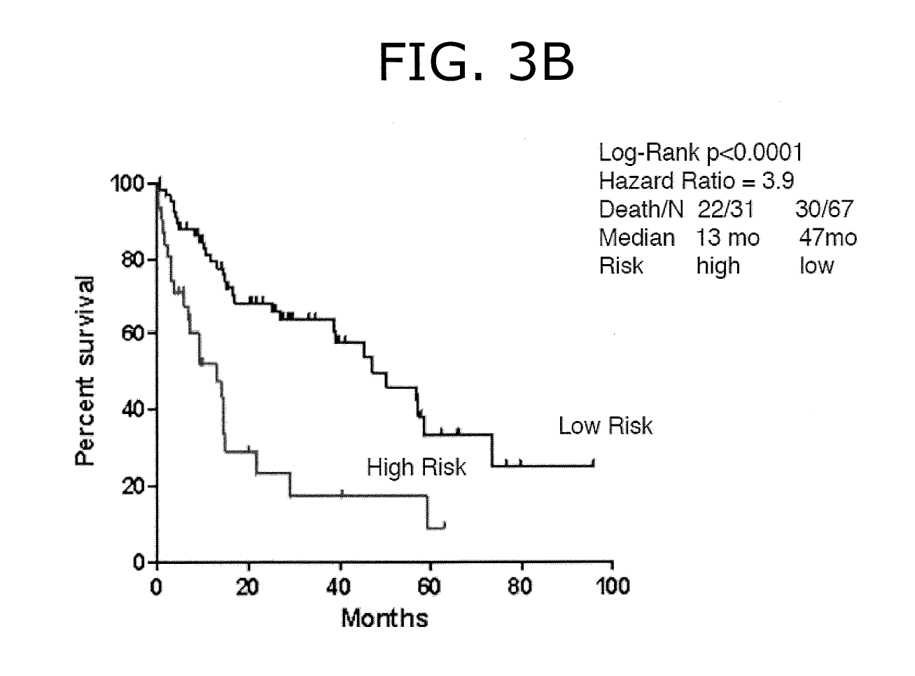

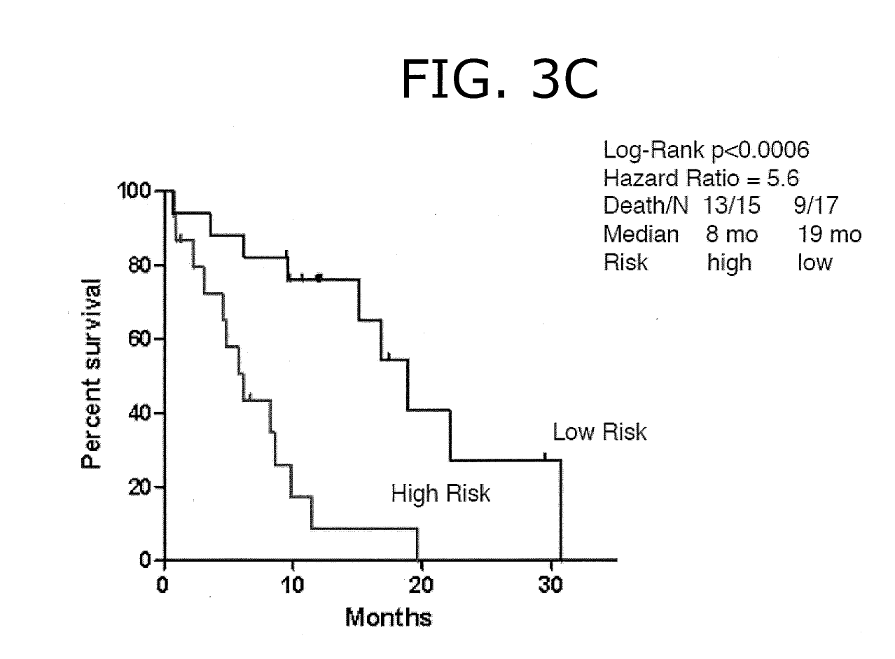

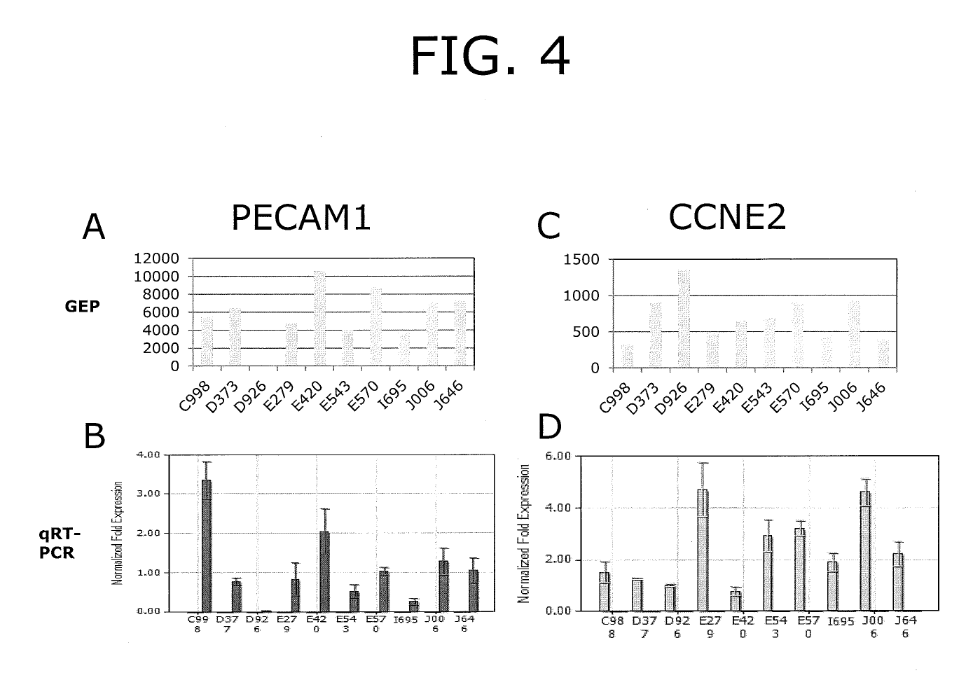

Since expression ratios do not reflect signal intensities, it also was determined whether expression signals of the 72 probe sets at relapse, each probe set dichotomized at the median, was associated with post relapse survival of the 127 TT2 patients. BRB ArrayTools identified 21 probesets (18 genes), significantly associated with survival, with a univariate p value of <0.05; the probe sets are listed in Table 5. BRB ArrayTools also used 20 of these probe sets (17 genes) to predict post relapse survival, with a hazard ratio of 4.4 (FIGS. 3A-3C).

TABLE 5
Genes whose expession is significantly associated with survival

| | Gene | Parametric | Permutation * | Hazard | Signal | |
|---|---|---|---|---|---|---|
| Probe set | symbol | p-value | p-value | Ratio^{#} | Median (range) | |
| 205034_at | CCNE2 | <1e−07 | <1e−07 | 1.828 | 575 | (167-4339) |
| 208982_at | PECAM1 | 2e−7 | <1e−07 | 0.635 | 9126 | (285-36351) |
| 208983_s_at | PECAM1 | 2.3e−6 | <1e−07 | 0.77 | 3638 | (18-15865) |
| 202768_at | FOSB | 4.8e−6 | <1e−07 | 0.757 | 1757 | (29-12913) |
| 208981_at | PECAM1 | 5.1e−5 | <1e−07 | 0.738 | 6947 | (134-15633) |
| 203665_at | HMOX1 | 8.2e−5 | <1e−07 | 0.721 | 869 | (83-22175) |
| 223961_s_at | CISH^{$} | 1.7e−4 | 1e−4 | 0.757 | 237 | (11-2705) |
| 201465_s_at | JUN | 8.2e−4 | 9e−4 | 0.782 | 1354 | (46-11121) |
| 229009_at | SIX5 | 0.001277 | 0.001 | 0.745 | 614 | (26-2971) |
| 201044_x_at | DUSP1 | 0.0023 | 0.0022 | 0.815 | 1122 | (10-7771) |
| 203821_at | HBEGF | 0.0046 | 0.0049 | 1.219 | 158 | (21-4625) |
| 206176_at | BMP6 | 0.0065 | 0.0069 | 0.735 | 4726 | (405-21385) |
| 219541_at | LIME1 | 0.0075 | 0.007 | 1.436 | 1287 | (80-4241) |
| 210538_s_at | BIRC3 | 0.011 | 0.0118 | 0.88 | 2760 | (21-23688) |
| 201041_s_at | DUSP1 | 0.015 | 0.0143 | 0.792 | 22829 | (1075-50162) |
| 222245_s_at | FER1L4 | 0.016 | 0.0173 | 0.712 | 1377 | (262-8790) |
| 227697_at | SOCS3 | 0.020 | 0.0216 | 1.108 | 111 | (3-3334) |
| 207001_x_at | TSC22D3 | 0.040 | 0.0396 | 0.839 | 1344 | (81-9652) |
| 36711_at | MAFF | 0.042 | 0.0494 | 1.189 | 405 | (8-9428) |
| 210845_s_at | PLAUR^{$} | 0.047 | 0.0468 | 0.782 | 430 | (45-1548) |
| 203068_at | KLHL21 | 0.048 | 0.0497 | 1.18 | 214 | (4-2134) |

* Permutation p-values for significant genes were computed based on 10000 random permutations.
^{#}Hazard ratio is the ratio of hazards for a two-fold change in the gene expression level.
^{$}Expression of these genes at relapse was lower than baseline, whereas their expression was higher after co-culture.

This set of genes from the 13 probeset model was further refined to the 11 genes and their chromosome locations shown in Table 6. Expression of 11 genes with a false discovery rate of ≦5% predicted post relapse survival with a hazard ratio of 8.4 and p<0.0001 of the 127 patients (FIG. 3A). Moreover, of the 127 TT2 patients, this model predicted post relapse survival of 32 relapsed patients following TT3 treatment (FIG. 3B), and of 98 patients who relapsed after achieving complete or near complete remission on several treatment regimens (FIG. 3C), 91 of them including high-dose therapy, suggesting that expression of the selected genes is universally associated with survival following relapse.

TABLE 6
11 genes (13 probesets) whose expression at
first relapse predicts post-relapse survival.

| Probeset | Gene Symbol | Chromosome location |
|---|---|---|
| 203665_at | HMOX1 | chr22q12\|22q13.1 |
| 203821_at | HBEGF | chr5q23 |
| 205034_at | CCNE2 | chr8q22.1 |
| 223961_s_at | CISH | chr3p21.3 |
| 201044_x_at | DUSP1 | chr5q34 |
| 201465_s_at | JUN | chr1p32-p31 |
| 202768_at | FOSB | chr19q13.32 |
| 206176_at | BMP6 | chr6p24-p23 |
| 208981_at | PECAM1 | chr17q23 |
| 208982_at | PECAM1 | chr17q23 |
| 208983_s_at | PECAM1 | chr17q23 |
| 219541_at | LIME1 | chr20q13.3 |
| 229009_at | SIX5 | chr19q13.32 |

A set of 33 probe sets, identified by coculture with osteoclasts, was found to predict post relapse survival. The 33 probe sets are given in Table 7

TABLE 7
33 Probe sets predictive of post-relapse survival

| | | | | | Chromosomal |
|---|---|---|---|---|---|
| No | Gene Id | Symbol | Weights (wᵢ)* | p-value | Location |
| 1 | 219073_s_at | OSBPL10 | −0.093019 | <1e−07 | chr3p22.3 |
| 2 | 205034_at | CCNE2 | 0.039816 | 1.00E−07 | chr8q22.1 |
| 3 | 211764_s_at | UBE2D1 | 0.019541 | 2.00E−07 | chr10q11.2-q21 |
| 4 | 208982_at | PECAM1 | −0.057215 | 1.50E−06 | chr17q23 |
| 5 | 203382_s_at | APOE | −0.019823 | 7.10E−06 | chr19q13.2 |
| 6 | 201443_s_at | ATP6AP2 | 0.015442 | 1.44E−05 | chrXp11.4 |
| 7 | 204944_at | PTPRG | 0.021603 | 1.82E−05 | chr3p21-p14 |
| 8 | 219540_at | ZNF267 | 0.018927 | 1.86E−05 | chr16p11.2 |
| 9 | 208983_s_at | PECAM1 | −0.088241 | 2.84E−05 | chr17q23 |
| 10 | 203665_at | HMOX1 | −0.003104 | 3.11E−05 | chr22q12\|22q13.1 |
| 11 | 216841_s_at | SOD2 | 0.022577 | 3.72E−05 | chr6q25.3 |
| 12 | 201266_at | TXNRD1 | 0.011841 | 3.79E−05 | chr12q23-q24.1 |
| 13 | 216942_s_at | CD58 | 0.032742 | 4.72E−05 | chr1p13 |
| 14 | 223344_s_at | MS4A7 | −0.002062 | 7.40E−05 | chr11q12 |
| 15 | 214146_s_at | PPBP | −0.036276 | 9.09E−05 | chr4q12-q13 |
| 16 | 200878_at | EPAS1 | −0.018851 | 1.39E−04 | chr2p21-p16 |
| 17 | 213746_s_at | FLNA | 0.052525 | 1.48E−04 | chrXq28 |
| 18 | 210753_s_at | EPHB1 | −0.052594 | 1.84E−04 | chr3q21-q23 |
| 19 | 206214_at | PLA2G7 | −0.003955 | 2.13E−04 | chr6p21.2-p12 |
| 20 | 223961_s_at | CISH | −0.066213 | 2.38E−04 | chr3p21.3 |
| 21 | 213112_s_at | SQSTM1 | −0.012277 | 2.66E−04 | chr5q35 |
| 22 | 228377_at | KLHL14 | −0.097634 | 2.90E−04 | chr18q12.1 |
| 23 | 202872_at | ATP6V1C1 | 0.014609 | 2.95E−04 | chr8q22.3 |
| 24 | 217047_s_at | FAM13A | −0.063961 | 3.12E−04 | chr4q22.1 |
| 25 | 208981_at | PECAM1 | −0.059574 | 3.53E−04 | chr17q23 |
| 26 | 224901_at | SCD5 | −0.072739 | 3.72E−04 | chr4q21.22 |
| 27 | 212737_at | GM2A | −0.018788 | 4.11E−04 | chr5q31.3-q33.1 |
| 28 | 228759_at | CREB3L2 | −0.045327 | 4.36E−04 | chr7q34 |
| 29 | 203645_s_at | CD163 | −0.009919 | 4.82E−04 | chr12p13.3 |
| 30 | 235706_at | CPM | −0.005803 | 5.01E−04 | chr12q14.3 |
| 31 | 205057_s_at | IDUA | −0.050522 | 5.09E−04 | chr4p16.3 |
| 32 | 219888_at | SPAG4 | −0.041312 | 5.64E−04 | chr20q11.21 |
| 33 | 210785_s_at | C1orf38 | −0.027797 | 5.85E−04 | chr1p35.3 |

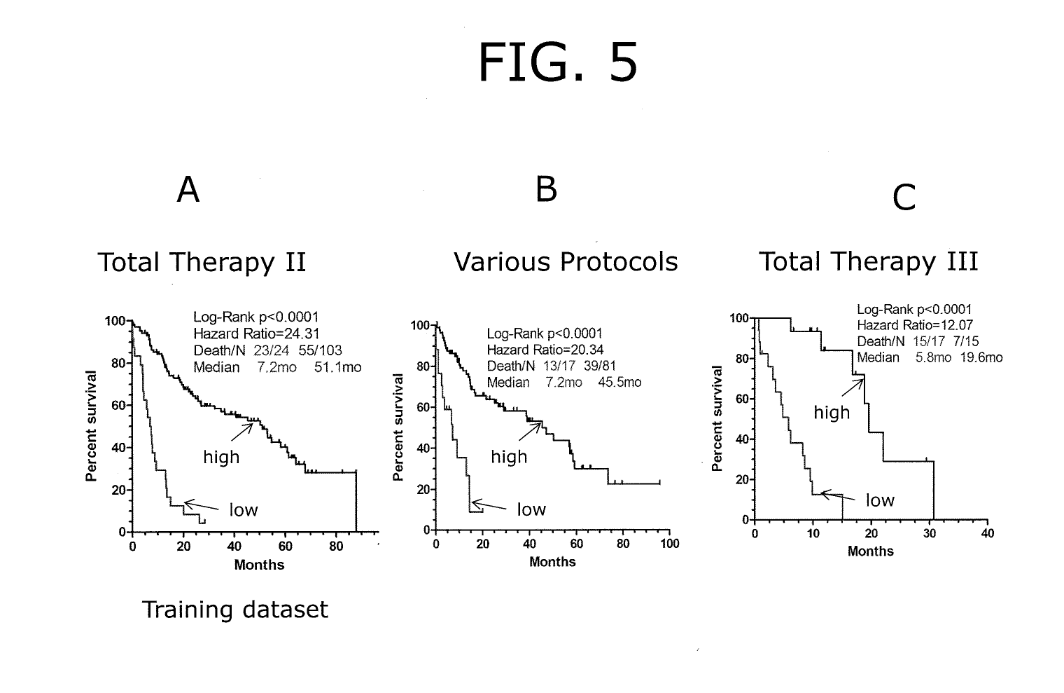

Post relapse survival rates for high and low risk patients from different groups evaluated by GEP and the 33 gene model in Table 7 are shown in FIGS. 5A-5C. A sample was predicted as high risk if its prognostic index was >0.794828, computed by the formula: PI=Σᵢwᵢxᵢ+6.999731, where wᵢ and xᵢ are the weight and logged gene expression for the i^{th} gene.

The following references are cited herein.
1. Barlogie et al., (2008) Cancer, 112:2720-2725.
2. Caldon, C. E. & Musgrove, E. A. (2010) Cell Div., 5:2.
3. Maerki et al., (2009) J Cell Biol, 187, 791-800.
4. Vallario et al., (1999) Human myeloma cells express the CD38 ligand CD31. British Journal of Haematology, 1999 May; 105:441-444.
5. Lu et al., (2009) Microvascular Research, 77:78-86.
6. Schmidmaier et al., (2006) Int J Biol Markers, 21:218-222.
7. Okada et al., (1999) Clin Exp Metastasis, 17:623-629.
8. Hjertner et al., (2000) Br J Haematol, 109:815-822.
9. Dong et al., (2000) The Journal of Immunology, 164:129-135.
10. Jarvenpaa et al., (2007) Gynecol Endocrinol., 23:351-355.
11. Pasqualini et al., (2000) Cancer Research, 60:722-727.
12. Bermudez et al., (2010) AJP-Cell Physiology, 299:C189-C202.
13. Chen et al., (2010) Blood, 115, 61-70.
14. Mittelstadt & Ashwell, (2001) Journal of Biological Chemistry, 276:29603-29610.
15. Brenner et al., (2004) Circulation Research, 94:1008-1010.
16. Diaz et al., (2005) Blood, 105:1237-1245.
17. Feng et al., (2008) Molecular Cancer Therapeutics, 7:1494-1505.
18. Nerini-Molteni et al., (2008) British Journal of Haematology, 141:494-503.
19. Katsuoka et al., (2005) Mol. Cell Biol, 25:8044-8051.
20. Massrieh et al., (2006) Antioxid. Redox. Signal., 8:53-59.
21. Motohashi et al., (2004) Proc Natl Acad Sci U.S.A, 101:6379-6384.
22. Yasukawa et al. (2000) Annu. Rev Immunol, 18:143-164.
23. Pham et al., (2005) J Cell Biochem, 95:990-1001.
24. Hoskins, et al., (2007) Am J Hum. Genet., 80:800-804.
25. Nonomura, et al., (2010) J Anat., 216:80-91.
26. Yajima et al., (2010) Exp Cell Res.
27. Seckinger et al., (2009) Oncogene, 28:3866-3879.
28. Ahn et al. (2006) Blood, 107:1521-1527.
29. Annunziata, et al., (2007) Cancer Cell, 12:115-130.
30. Petersen et al., (2010) Proc Natl Acad Sci U.S.A, 107:11936-11941.
31. Rigolin et al., (2003) Br J Haematol, 120:953-959.
32. Barlogie et al., (2006) The New England Journal of Medicine, 354:1021-1030.
33. Barlogie et al., (2008) Blood, 112:3115-3121.
34. Nair et al., (2009) Blood, 113:6572-6575.
35. Zhan et al., (2002) Blood, 99:1745-1757.
34. Mesenchymal Stem Cells: Methods and Protocols. Prockop, et al., [449]. 2008. Humana Press. Methods in Molecular Biology. Walker, J.
36. Yaccoby et al., (2004) Cancer Res., 64:2016-2023.
37. Cox et al. (1972) Journal of the Royel Statistical Society B, 34:187-220.
38. Korn et al., (2004) Journal of Statistical Planning and Inferences, 124:379-398.
39. Simon et al., (2003) Design and Analysis of DNA Microarray Investigations, Springer-Verlag, New York.

While the invention has been described with reference to certain particular embodiments, those skilled in the art will appreciate that various modifications may be made without departing from the spirit and scope of the invention.

All patents and publications mentioned in this specification are indicative of the level of those skilled in the art to which the invention pertains. All patents, publications, and sequence references (such as gene names and reference acesison numbers, including their underlying human gene sequences and associated annotations in databases such as the NCBI Entrez portal) herein are incorporated by reference to the same extent as if each individual reference was specifically and individually indicated as having been incorporated by reference in its entirety. Where there is any conflict between a reference incorporated by reference and this application, this application will control.

Headings used in this application are for convenience only and do not affect the interpretation of this application.

1. A method for predicting post-relapse survival of a relapsed multiple myeloma patient, comprising testing the level of gene expression of a group of multiple myeloma genes in multiple myeloma plasma cells obtained from the patient, thereby predicting the post-relapse survival of the patient, wherein the group of multiple myeloma genes comprises at least CCNE2, PECAM1, HMOX1, and CISH;
optionally wherein the group of multiple myeloma genes further comprises HBEGF, JUN, SIX5, and DUSP1;
optionally wherein the group of multiple myeloma genes further comprises BMP6, FOSB, and LIME1.

2. The method of claim 1, wherein:
a decrease in expression of PECAM1, HMOX1, CISH, SIX5, BMP6, JUN, FOSB, and/or DUSP1; and/or
an increase in expression of LIME1, CCNE2, and/or HBEGF, is predictive of a low survival rate of the patient.

3. The method of claim 1, wherein the group of multiple myeloma genes further comprises BIRC3, FER1L4, TSC22D3, MAFF, SOCS3, and KLHL21.

4. The method of claim 3, wherein:
a decrease in the expression level of BIRC3, FER1L4, and/or TSC22D3; and/or
an increase in expression level of MAFF, SOCS3 and/or KLHL21, is predictive of a low survival rate of the patient.

5. A method for predicting post-relapse survival of a relapsed multiple myeloma patient, comprising testing the level of gene expression of a group of multiple myeloma genes in plasma cells obtained from the patient, thereby predicting the post-relapse survival of the patient, wherein the multiple myeloma genes comprises all of OSBPL10, CCNE2, UBE2D1, PECAM1, APOE, ATP6AP2, PTPRG, ZNF267, HMOX1, SOD2, TXNRD1, CD58, MS4A7, PPBP, EPAS1, FLNA, EPHB1, PLA2G7, CISH, SQSTM1, KLHL14, ATP6V1C1, FAM13A, SCD5, GM2A, CREB3L2, CD163, CPM, IDUA, SPAG4, and Clorf38.

6. The method of claim 5, wherein the method comprises determining a disease index that is a weighted sum the levels of gene expression for the multiple myeloma genes.

7. The method of claim 6, wherein the levels of gene expression are log transformed.

8. The method of claim 7, wherein the weights for the logged gene expression levels are about the weights given in Table 7.

9. The method of claim 8, wherein a disease index above about 0.79 indicates that the subject has a low survival rate, where the disease index is calculated by the formula: DI=Σᵢwᵢxᵢ+6.999731, where wᵢ and xᵢ are the weight and logged gene expression for the i^{th} gene.

10. A method for predicting post-relapse survival of a relapsed multiple myeloma patient, comprising testing the level of gene expression of a group of multiple myeloma genes in plasma cells obtained from the patient, thereby predicting the post-relapse survival of the patient, wherein the group of multiple myeloma genes comprises all of AMPD1, ANPEP, BIRC3, BMP6, BTLA, CBFA2T3, CCNE2, CCR2, CCR7, CD44, CD48, CD82, CEP170, CHST11, CISH, CITED2, DUSP1, EPB41L3, FAM129B, FER1L4, FOS, FOSB, GLA, GPRC5D, HBEGF, HMOX1, ICAM1, IFI30, IL8, ISG20, JUN, KANK1, KLF2, KLHL21, LIME1, LRRN1, MAFF, MMP14, MT1E, MT1H, PECAM1, PLAC8, PLAU, PLAUR, PRRG4, SAT1, SGK1, SIX5, SLC25A19, SLC2A4RG, SOCS3, SPHK1, TGM2, TMEM49, TNFAIP2, TNFRSF17, TSC22D3, and UGT2B17.

11. The method of claim 1, wherein the plasma cells are CD138+.

12. The method of claim 1, wherein testing the level of gene expression of the genes in the group of multiple myeloma genes comprises:
hybridizing nucleic acids obtained from the multiple myeloma plasma cells to one or more platforms comprising probe sets hybridizable to one or more genes in the group of multiple myeloma genes; and
converting intensity of a signal generated upon hybridization to the value of gene expression for each multiple myeloma gene in the group.

13. The method of claim 1, wherein the level of gene expression is measured using an oligonucleotide array.

14. The method of claim 13, wherein the oligonucleotide array is a U133Plus2.0 microarray.

15. The method of claim 1, wherein the level of gene expression is measured by polymerase chain reaction (including quantitative PCR, real-time PCR, and quantitative real-time PCR (qRT-PCR)) or sequencing.

16. A method of treating a relapsed multiple myeloma patient, comprising administering a suitable multiple myeloma therapy to patient evaluated by claim 1.

17. The method of claim 16, wherein the patient is predicted to have a low rate of survival and the multiple myeloma therapy administered to the patient is a salvage therapy.

18. (canceled)
